(19) United States
(12) Patent Application Publication
(10) Pub. No.: US 2024/0058470 A1
(43) Pub. Date: Feb. 22, 2024

(54) POLYMER-CONJUGATED MICROBUBBLES FOR HIGH DRUG/GENE LOADING

(71) Applicant: THE BOARD OF REGENTS OF THE UNIVERSITY OF TEXAS SYSTEM, AUSTIN, TX (US)

(72) Inventors: JACQUES LUX, PLANO, TX (US); SINA KHORSANDI, DALLAS, TX (US); CAROLINE DE GRACIA LUX, PLANO, TX (US); ROBERT F. MATTREY, DALLAS, TX (US)

(73) Assignee: THE BOARD OF REGENTS OF THE UNIVERSITY OF TEXAS SYSTEM, AUSTIN, TX (US)

(21) Appl. No.: 18/248,862

(22) PCT Filed: Oct. 13, 2021
(86) PCT No.: PCT/US2021/054820

§ 371 Date: Apr. 12, 2023

Related U.S. Application Data
(60) Provisional application No. 63/091,204, filed on Oct. 13, 2020.

(51) Int. Cl. A61K 47/69; A61K 41/00

(52) U.S. Cl. CPC A61K 47/6925; A61K 41/0028

(57) ABSTRACT

Spermine-decorated microbubbles and methods of making the same as well as methods for using spermine-decorated microbubbles for drug delivery are described.

### CROSS-REFERENCE TO RELATED APPLICATIONS

This application claims the benefit of priority to U.S. Provisional Application No. 63/091,204, filed on Oct. 13, 2020, the entire contents of which are hereby incorporated herein by reference.

### FIELD OF THE DISCLOSURE

The present disclosure relates to microbubble compositions for effecting drug delivery via microbubbles and methods of making and using microbubble compositions, particularly in combination with sonoporation.

### BACKGROUND

Drug delivery often involves the use of a carrier to deliver a drug or therapeutic agent (e.g., a gene or therapeutic protein) to cells and optionally to induce the uptake of the therapeutic agent by the cells (e.g., transfer across the cell plasma membrane). Drug delivery carriers may additionally serve to target specific cell types and may improve the bioavailability of a therapeutic agent to specific tissues for a drug delivered systemically in vivo. Drug delivery faces many challenges in achieving high drug/carrier loading efficiencies, stable loading, protection of the therapeutic agents from biodegradation, targeting specific tissues and cell types, and promoting efficient uptake of the drug by the cells. Gene therapy, in particular, holds the promise of providing single treatment curative benefits for disease. The majority of clinical gene therapies make use of viral vectors, which are limited by potential mutagenesis, immunogenicity, and non-specific delivery. Microbubbles are clinically used ultrasound contrast agents that have been considered as promising vehicles for targeted drug delivery. However, reports of drug delivery utilizing cationic microbubbles have shown only weak and unstable binding to payloads such as DNA, and also identify concerns about unacceptable toxicity.

### SUMMARY

Disclosed herein are microbubble compositions for effectively loading large amounts of payload, such as nucleic acids or other pharmaceutical deliverables, onto microbubbles for drug delivery (e.g., gene therapy applications). Kits comprising microbubble compositions, methods of making microbubble compositions, and methods of using microbubble compositions for drug delivery are also disclosed herein. The external surface of the microbubble compositions may be decorated with spermine molecules for effectively binding to a payload. Large amounts of spermine molecules may be associated with the microbubbles via interlinking polymers, such as dextran, which can link one or more spermine molecules to the microbubbles. The payload may be effectively delivered across cell membranes in a spatially and temporally targeted manner using sonoporation techniques.

In one aspect of the disclosure, a microbubble composition for delivering a payload to one or more cells comprises a plurality of spermine-decorated microbubbles. The microbubbles each comprise a gas core encapsulated by a surfactant shell and a plurality of spermine molecules are associated with the external surface of the surfactant shell of each microbubble.

The plurality of spermine molecules may be non-covalently coupled to the external surface of each microbubble. The plurality of spermine molecules may be covalently coupled to the external surface of each microbubble. The plurality of spermine molecules may be directly coupled to the external surface of each microbubble. The plurality of spermine molecules may be indirectly coupled to the external surface of each microbubble by an interlinking polymer. The interlinking polymer may be covalently crosslinked to the external surface of each microbubble by one or more spermine molecules.

The interlinking polymer may be a scaffold polymer having multiple spermine molecules of the plurality of spermine molecules covalently coupled to each scaffold polymer. The scaffold polymer may be linear. The scaffold polymer may be branched. The scaffold polymer may be dextran. The dextran may have an average molecular weight of at least 5 kDa, at least 10 kDa, between about 20 kDa and 50 kDa, or between about 35 kDa and 45 kDa. The average molecular weight may be about 40 kDa.

The gas core may comprise a perfluorocarbon. The perfluorocarbon may be decafluorobutane. The surfactant shell may comprise lipids. The lipids may be phospholipids. The phospholipids may comprise one or both of 1,2-Distearoyl-sn-Glycero-3-Phosphocholine (DSPC) and 1,2-Distearoyl-sn-Glycero-3-Phosphoethanolamine (DSPE) lipids. The surfactant shell comprises PEGylated molecules. The surfactant shell may comprise a plurality of reactive groups exposed on the external surface of the surfactant shell. The reactive groups may be maleimides. The reactive groups may be linked to the surfactant molecules by a PEG linker. The interlinking polymer may be coupled to the microbubble via a biodegradable linker, which may optionally be cleavable at a pH inside a lysosome.

The plurality of spermine molecules on each microbubble may be associated with a plurality of payload molecules. The payload molecules may comprise proteins. The payload molecules may comprise nucleic acids. The nucleic acids may comprise DNA, which may optionally be a plasmid. The microbubble may be loaded with at least about 30,000 nucleic acids per microbubble or at least about 0.025 μg/μm 2 of nucleic acid.

The microbubbles may further comprise targeting molecules on the external surface of the surfactant shell configured to bind the one or more cells. The targeting molecules may be antibodies.

The average microbubble size of the microbubble composition is between about 1 μm and 10 μm, between about 1 μm and about 5 μm, or about 3 μm.

The plurality of spermine molecules may comprise polyspermines.

One or more spermines of the spermine-decorated microbubbles may be linked to another spermine or to an interlinking polymer by a biodegradable linker. The biodegradable polymer may be cleavable at a pH inside a lysosome. The biodegradable linker may comprise cystamine bisacrylamide or bisacrylamide ketal.

At least about 5%, 10% 15%, 20%, 25%, or 50% of the microbubbles in the composition may be crosslinked to another microbubble via a spermine. Alternatively, no more than about 5%, 10% 15%, 20%, 25%, or 50% of the microbubbles in the composition may be crosslinked to another microbubble via a spermine.

In another aspect of the disclosure, a method of making a microbubble composition for delivering a payload to one or more cells comprises mixing a solution comprising spermine molecules with a solution of microbubbles. The microbubbles each comprise a gas core encapsulated by a surfactant shell. The method may be a method for making any one of the microbubble compositions described above.

The solution comprising spermine molecules may comprise spermines conjugated to polymers. The polymers may be scaffold polymers having multiple spermine molecules covalently coupled to the scaffold polymer. The scaffold polymer may be linear or branched. The scaffold polymer may be dextran. The solution comprising spermine molecules may be made with dextran having an average molecular weight of at least 5 kDa, at least 10 kDa. between about 20 kDa and 50 kDa, or between about 35 kDa and 45 kDa. The solution comprising spermine molecules may comprise comprises polyspermines. A plurality of spermines within the polyspermines may be linked by a biodegradable linker.

The microbubble solution may be gradually added into the solution comprising spermine molecules to allow for saturation of the microbubbles with the spermine molecule, optionally during the mixing process. The solution comprising spermine molecules may be gradually added into the microbubble solution to promote crosslinking of the microbubbles via the spermine molecules.

The solution comprising spermine molecules may comprise a plurality of first reactive groups and the microbubble solution may comprise a plurality of second reactive groups exposed on an external surface of the surfactant shells of the microbubbles. The first and second reactive groups may be configured to covalently couple the spermine molecules to the microbubbles. The first reactive group may be a thiol and the second reactive group may be a maleimide. The mixture of the solution comprising spermine molecules and the microbubble solution may comprise a molar ratio of first reactive groups to second reactive groups of at least 2:1, at least 5:1, at least 10:1, or at least 20:1. The microbubble solution may comprise a molar ratio of second reactive group molecules to surfactant molecules of about 1:20. The microbubble solution may comprise between 1×10⁸ and 1×10¹⁰ microbubbles/mL. The microbubble solution may comprise approximately 1.10×10⁹ microbubbles/mL.

The method may further comprise mixing in a solution comprising payload molecules. The payload solution may be mixed with the solution comprising spermine molecules prior to mixing with the microbubble solution. The solution comprising spermine molecules may be mixed with the microbubble solution prior to mixing with the payload solution. The solution comprising microbubbles may be gradually added into the payload solution to allow for saturation of the microbubbles with the payload molecules, optionally while mixing. The payload molecules may comprise proteins. The payload molecules comprise nucleic acids. The nucleic acids may comprise DNA, which may optionally be a plasmid. The mixture of the solution comprising microbubbles and the payload solution may comprise a ratio of payload mass to microbubble number of at least 1×10⁻⁸ μg/microbubble, optionally at least 2×10⁻⁸ μg/microbubble, and/or wherein the mixture comprises a spermine amine:payload phosphate (N:P) ratio of about 1:1, 1:2, 1:5, or 1:10. The method of making microbubbles may produce a microbubble composition having on average at least 5,000; at least 10,000; at least 20,000; or at least 30,000 payload molecules/microbubble. The method may produce a spermine-dextran decorated microbubble composition having at least 1.0×10⁻¹⁴ g of spermine-dextran per microbubble.

The method may further comprise mixing a solution comprising targeting molecules with a solution comprising the microbubbles. The solution comprising targeting molecules may be the same solution as the solution comprising spermine molecules. The solution comprising targeting molecules may be the same solution as the payload solution. The solution comprising targeting molecules may be added to the microbubble solution after the solution comprising spermine molecules or before the solution comprising spermine molecules. The targeting molecules may be antibodies.

In another aspect of the disclosure, disclosed are microbubbles compositions produced by any of the aforementioned methods of making microbubbles.

In another aspect of the disclosure, a method of delivering a payload to one or more cells using sonoporation comprises exposing the one or more cells to a plurality of spermine-decorated microbubbles and then exposing the one or more cells to an ultrasound stimulus configured to sonoporate the one or more cells. The ultrasound may be delivered at about 1-2 W/cm², optionally with 50% duty cycle. The ultrasound may be delivered for between about 30-60 seconds. The cells may be exposed to the microbubbles for at least about 10 minutes prior to delivering ultrasound stimulus. The method may further comprise using ultrasound to visualize the microbubbles prior to delivering the ultrasound stimulus configured to sonoporate the one or more cells. The intensity of the ultrasound used to visualize the microbubbles may be less than the intensity of the ultrasound stimulus.

The method may be an in vivo method in which exposing the one or more cells to the plurality of microbubbles comprises administering a composition comprising the plurality of microbubbles to a subject.

The method may be an in vitro method. The plurality of microbubbles may be incubated with the one or more cells at a concentration of at least about 5, 10, 15, 20, 25, or 30 microbubbles/cell. The plurality of microbubbles may be incubated with the one or more cells at a concentration between about 15 and about 20 microbubbles/cell. The plurality of microbubbles may be mixed together with the one or more cells. The plurality of microbubbles may be provided by any of the microbubble compositions described above.

In another aspect of the disclosure is a kit containing any of the microbubble compositions described above, and optionally a composition of payload molecules for mixing with the microbubble composition.

### BRIEF DESCRIPTION OF THE DRAWINGS

FIG. 1 depicts the chemical structure of spermine, including the pKa of the molecule's amino groups.

FIG. 2 depicts the chemical structure of polyethylenimine (PEI).

FIG. 3 depicts the chemical structure of branched dextran.

FIG. 4 depicts the chemical oxidation of dextran and the chemical synthesis spermine-dextran from spermine and oxidized dextran.

FIG. 5 depicts the NMR spectra obtained from a synthesis of oxidized dextran (40 kDa) (top) and spermine-dextran (bottom).

FIG. 6 depicts particle size measurements of spermine-dextran/pDNA polyplexes at N:P ratios of 1:1 (top-left), 1:2 (top-right), 1:5 (bottom-left) and 1:10 (bottom-right).

FIG. 7 depicts an agarose gel electrophoresis experiment on spermine-dextran/pDNA polyplexes loaded at N:P ratios of 1:1, 1:2, 1:5, and 1:10 against controls of unloaded spermine-dextran as well as pDNA alone.

FIG. 8 depicts the measured pDNA loading capacity in terms of the number of DNA molecules per microbubble for spermine-dextran microbubbles prepared according to the present disclosure compared relative to DSTAP microbubbles known in the art.

FIG. 9 depicts flow cytometry results for control JeKo cells (top) and sonoporated Jeko cells (bottom) which were incubated with spermine-dextran-ROR1 microbubbles loaded with pDNA.

FIG. 10 depicts an agarose gel electrophoresis experiment of sonoporated JeKo cells transfected with spermine-dextran-ROR1 microbubbles loaded with pDNA and incubated with the Jeko cells at approximately 5 microbubbles per cell (left) and 15 microbubbles per cell (right) and sonoporated at 2 W/cm² for 5 s, 2 W/cm² for 30 s, 1 W/cm² for 60 s, or 2 W/cm² for 60 s, or not sonoporated at all (negative control).

FIG. 11 depicts the chemical synthesis of TFA₂-spermine.

FIG. 12 depicts the chemical synthesis of BOC₂-spermine.

FIG. 13 depicts the chemical synthesis of poly diaminoethane (polyDAE).

FIG. 14 depicts the chemical synthesis of polyspermine.

FIG. 15 depicts the chemical synthesis of bisacrylamide ketal.

FIG. 16 depicts the chemical synthesis of phthalimde-spermine.

FIG. 17 depicts the chemical synthesis of polyspermine CBA.

### DETAILED DESCRIPTION

### Microbubble Compositions

### Microbubbles

As used herein, “microbubble” may refer to a bubble formed by a surfactant shell encapsulating a gas core. The surfactant shell may comprise one or more types of molecules, which lower the interfacial tension between the gas core and the exterior aqueous environment, such as a physiological environment. The shell may comprise, for example, lipids (e.g., phospholipids), proteins (e.g., albumin), sugars, and/or polymers. In some embodiments, the bubble may be no greater than about 10 μm in diameter. Unless otherwise specified, microbubbles, as used herein, may include bubbles less than 1 μm (i.e. nanobubbles), such as bubbles between, for example, about 100 nm-1 μm, about 200 nm-1 μm, or about 300 nm-1 μm. In some embodiments, the average microbubble size within a microbubble composition is at least about 1, 2, 3, 4, or 5 μm. In some embodiments, the average microbubble size is approximately 1, 2, 3, 4, or 5 μm. In some embodiments, the average microbubble size is between approximately 1-10, 1-5, 1-4, 1-3, 1-2, 2-5, 2-4, 2-3, 3-5, or 3-4 μm.

The gas core may comprise one or more gases. In some embodiments, the gas core comprises one or more perfluorocarbons. The one or more perfluorocarbons may comprise octafluoropropane (OFP)/perfluoropropane (PFP), decafluorobutane (DFB)/perfluorobutane (PFB), Dodecafluoropentane (DDFP)/perfluoropentane/perflenapent, tetradecafluorohexane/perfluorohexane, or hexadecafluoroheptane/perfluoroheptane, octadecafluorodecalin/perfluorodecalin, or perfluoro(2-methyl-3-pentanone) (PFMP). In some embodiments, the gas core may comprise one of more of the following fluorocarbons: 1,2-(F-alkyl)ethenes; 1,2-bis(F-butyl)ethenes; 1-F-isopropyl, 2-F-hexylethenes 1,2-bis(F-hexyl)ethenes; perfluoromethyldecalins; perfluorodimethyldecalins; perfluoromethyl- and dimethyl-adamantanes, perfluoromethyl-, dimethyl- and trimethyl-bicyclo (3,3,1) nonanes and their homologs; perfluoroperhydrophenanthrene; ethers of formulae: (CF₃)₂CFO(CF₂ CF₂)₂OCF(CF₃)₂, (CF₃)₂CFO(CF₂ CF₂); OCF(CF₃)₂, (CF₃)₂CFO(CF₂ CF₂)₂F, (CF₃)₂CFO(CF₂ CF₂)₃F, F[CF(CF₃)CF₂O]₂CHFCF₃, [CF₃CF₂CF₂(CF₂)u]₂O with u=1, 3 or 5, and amines N(C₃F₇)₃, N(C₄F₉)₃, and N(C₅F₁₁)₃; perfluoro-N-methylperhydroquinolines and perfluoro-N-methylperhydroisoquinolines, and perfluoralkyl hydrides, such as C₆F₁₃H, C₈F₁₇H, C₈F₁₆H₂ and the halogenated derivatives C₆F₁₃Br, (perflubron), C₆F₁₃ CBr₂CH₂Br, 1-bromo 4-perfluoroisopropyl cyclohexane, C₈F₆Br₂, and CF₃O(CF₂CF₂O)ᵤCF₂CH₂OH with u=2 or 3. In some embodiments, the gas core comprises air. In some embodiments, the gas core comprises sulfur hexafluoride. In some embodiments, the gas core comprises nitrogen. Higher molecular weight gases may generally form more stable microbubbles than lower molecular weight gases.

In some embodiments, the surfactant shell may comprise lipids, such as phospholipids, which self-align under certain conditions to form a hydrophilic external surface and a lipophilic or hydrophobic internal surface. The phospholipids may comprise any standard phospholipid used in the art for forming microbubbles, nanodroplets, micelles, liposomes, etc. In some embodiments, the phospholipids may comprise diacylglyceride structures, such as phosphatidic acid (phosphatidate) (PA), phosphatidylethanolamine (cephalin) (PE), phosphatidylcholine (lecithin) (PC), phosphatidylserine (PS), and phosphoinositides (e.g., posphatidylinositol (PI), phosphatidylinositol phosphate (PIP), phosphatidylinositol bisphosphate (PIP2) and phosphatidylinositol trisphosphate (PIP3). In some embodiments, the phospholipids may comprise phosphosphingolipids, such as ceramide phosphorylcholine (Sphingomyelin) (SPH), ceramide phosphorylethanolamine (Sphingomyelin) (Cer-PE), and ceramide phosphoryllipid. In some embodiments, the phospholipid comprises 1,2-Distearoyl-sn-Glycero-3-Phosphocholine (DSPC) or derivatives thereof. In some embodiments, the phospholipid comprises 1,2-Distearoyl-sn-Glycero-3-Phosphoethanolamine (DSPE) or derivatives thereof. In some embodiments, the surfactant molecules may be coupled to polymer chains, such as poly(ethylene glycol) (i.e. the surfactant shell/microbubble may be PEGylated). For example, the surfactant molecule may comprise 1,2-Distearoyl-sn-Glycero-3-Phosphoethanolamine-N-[Methoxy(Polyethylene glycol)-2000] (DSPE-PEG2k). PEGylation of the microbubble may improve anti-flocculation/colloidal stability, anti-immunogenicity, hydrophilicity, biocompatibility, and/or in vivo circulation time/bioavailability of the microbubbles. PEGylation of the external surface of the microbubble may provide favorable conditions (e.g., steric) for performing conjugations which functionalize the microbubble surface. In some embodiments, the surfactant shell may comprise two types of surfactant molecules. In some embodiments, the surfactant shell may comprise three types of surfactant molecules. In some embodiments, the surfactant shell may comprise more than three types of surfactant molecules. In some embodiments, approximately 0-25%, 0-20%, 0-15%, 0-10%, 0-5%, 5-10%, 10-15%, 15-20%, 20-25%, 0-10%, 10-20%, 0-15%, or 15-25% of the surfactant molecules are PEGylated. For example, approximately 10% of the surfactant molecules may be PEGylated. In some embodiments, approximately 0-25%, 0-20%, 0-15%, 0-5%, 5-10%, 10-15%, 15-20%, 20-25%, 0-10%, 10-20%, 0-15%, or 15-25% of the surfactant molecules comprise functional groups configured to be exposed on the external surface of the surfactant shell (e.g., for linking to spermine molecules or targeting molecules). For example, in some embodiments, approximately 5% of the surfactant molecules are functionalized. In some embodiments, approximately half of the PEGylated surfactant molecules may comprise functional groups.

In some embodiments, the surfactant molecule may contain a functional group that is reactive in a conjugation reaction configured to covalently couple additional molecules to the microbubble shell. The functional group may be selected from any standard reactive group commonly used in the art to perform bioconjugations. For example, the functional group may comprise an amine, an isothiocyanate, an acyl azide, an NHS ester, a sulfonyl chloride, an aldehyde, a ketone, a glyoxal, an epoxide, an oxirane, a carbonate, an arylating agent, an imidoester, a carbodiimide, an anhydride, a fluorophenyl ester, a hydroxymethyl phosphine derivative, a guanadino group, a thiol, a haloacetyl or alkyl halide derivative, a maleimide, an aziridine, an acryloyl derivative, a pyridyl disulfide, a TNB-thiol, a disulfide reductant, a vinylsulfone derivative, a diazoalkane or diazoacetyl compound, a carbonyldiimidazole, a hydroxyl, an isocyanate, an aldehyde, a ketone, a hydrazine derivative, a Schiff base, a diazonium derivative, a benzophenome, an anthraquinone, a diazirine derivative, a psoralen compound, a boronic acid derivative, or a functional group that reacts with any of the foregoing. In some embodiments, the functional group may be one that partakes in a click reaction, such as a copper(I)-catalyzed azide-alkyne cycloaddition (CuAAC), a strain-promoted azide-alkyne cycloaddition (SPAAC), a strain-promoted alkyne-nitrone cycloaddition (SPANC), a strained alkene and azide [3+2] cycloaddition, a strained alkene and tetrazine inverse-demand Diels-Alder reaction, a strained alkene and tetrazole photoclick reaction, etc. In some embodiments, the functional group may be configured to partake in a non-covalent reaction. For example, the functional group may comprise streptavidin or biotin, an antibody or antigen, or a receptor or ligand. The functional group may be presented on the external surface of the surfactant shell. The functional group may be directly conjugated to the surfactant molecule or may be separated by a linker, such as a PEG linker. For example, the surfactant shell may comprise 1,2-Distearoyl-sn-Glycero-3-Phosphoethanolamine-N-Malemide(Polyethylene glycol)-2000].

Microbubbles of the microbubble compositions describe herein may generally be formed according to any process known in the art. In some embodiments, microbubbles are produced by sonication. In some embodiments, microbubble are produced by shaking. In some embodiments, microbubbles are produced from high pressure emulsification. In some embodiments, microbubbles are produced by activating the phase-transition of liquid-core nanodroplets. Microbubbles may be produced, for example, by any of the methods disclosed in U.S. Pat. No. 6,113,919 to Reiss et al. (issued Sep. 5, 200) or U.S. Patent Application Publication Nos. US 2002/0150539 to Unger (Oct. 17, 2002); US 2013/0336891 to Dayton et al. (published Dec. 19, 2013); US 2018/0272012 to de Gracia Lux et al. (published Sep. 27, 2018), each of which is hereby incorporated by reference in its entirety.

### Spermine Decoration

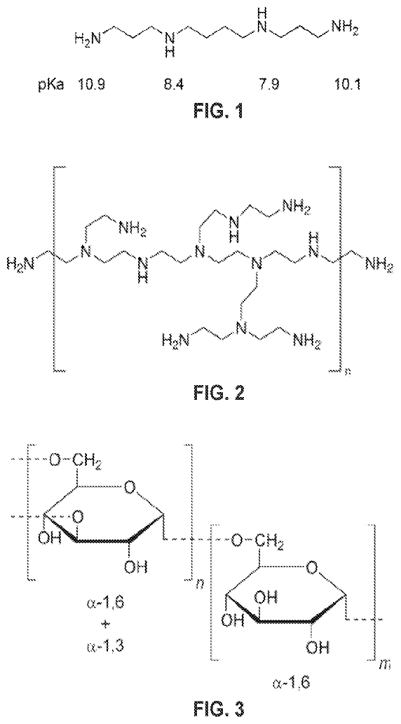

Spermine (1,12-diamino-4,9-diazadodecane, also known as N,N′-bis(3-aminopropyl)butane-1,4-diamine) is a polyamine, specifically an organic dialkylamine, involved in cellular metabolism that is found in all eukaryotic cells. The precursor for synthesis of spermine is the amino acid ornithine. It is an essential growth factor in some bacteria. It is found as a polycation at physiological pH. Spermine is associated with nucleic acids and is thought to stabilize helical structure, particularly in viruses. The amino groups of a spermine molecule have pKa's of approximately 10.9, 8.4, 7.9, and 10.1, as shown in FIG. 1. Spermine generally is fully protonated in aqueous environments at physiological pH (i.e. pH 7.4). By way of comparison, branched polyethylenimine (PEI), depicted in FIG. 2, is reported to have pKa values between 7.9 and 9.6 and at least 50% of the amine groups are generally expected to be protonated at physiological pH.

Spermine, as used herein, may refer to a spermine molecule as depicted in FIG. 1, or to a derivative or analogue thereof (e.g., thermospermine), including molecules synthesized from spermine, and spermidine (a precursor to spermine synthesis), all being encompassed by the term “spermine molecule”. Molecules synthesized from spermine can include polyspermines, which may comprise two or more spermines covalently linked together, possibly with intermediary crosslinkers. In the embodiments of the present disclosure, a spermine-like molecule which substantially resembles or is modeled off of spermine (e.g., polyDAE) may be substituted for a spermine molecule, if it achieves substantially the same effects as described elsewhere herein.

In some embodiments, polyspermines may be produced by polymerizing spermines together, possibly with intermediary crosslinkers. In some embodiments, multiple spermines may be linked together via the secondary amines depicted in FIG. 1, such that one or both of the primary amines depicted in FIG. 1 are left free after the reaction. In some embodiments, one or more of these primary amines may subsequently be used to link the spermine molecule to the microbubble or to an interlinking polymer, as described elsewhere herein. In some embodiments, the primary amines of a spermine molecule may be reacted with a protecting group such as 9-fluorenylmethoxycarbonyl (Fmoc), tert-Butyloxycarbonyl (Boc), ethyl trifluoroacetate, or a phthalimide. Protecting groups may be used to protect the primary amines of the spermine molecules when synthesizing larger molecules such as polyspermines or polymers comprising spermines. The protecting group may be removed with use of a standard agent known in the art to allow the primary amino groups to partake in additional reactions (e.g., conjugations to interlinking polymers and/or the microbubble) or to electrostatically bind to payload molecules, as described elsewhere herein.

In some embodiments, polyspermines may be formed (e.g., polymerized) with biodegradable/cleavable linkers between two or more of the spermine groups. Biodegradable linkers are generally known in the art, and include, for example, the linkers described in U.S. Pat. No. 8,580,545 to Alferiev et al. (issued Nov. 12, 2013), which is hereby incorporated by reference in its entirety. Biodegradable linkers may be configured to be cleaved (e.g., hydrolyzed) in specific physiological environments (e.g., low pH) and/or after a predictable amount of time within a physiological environment. For example, a spermine molecule may comprise polyspermine CBA (see, e.g., Example 23), which comprises spermines interlinked by a degradable cystamine bisacrylamide, which is prone to be reduced/cleaved at pH's below a physiological pH of 7.4. As another example, the spermine molecule may comprise spermines interlinked by a biodegradable bisacrylamide ketal (see, e.g., Example 21).

The microbubbles described herein in may be decorated with spermine molecules. Compositions of spermine-decorated microbubbles will generally comprise microbubbles in which a plurality of spermine molecules are associated with the external surface of the surfactant shell of each or substantially each microbubble within the composition. In some embodiments, each microbubble may be decorated with approximately 100,000, 200,000, 300,000, 400,000, 500,000, 600,000, 700,000, 800,000, 900,000, 1,000,000, 2,000,000, 3,000,000, 4,000,000, 5,000,000, 6,000,000, 7,000,000, 8,000,000, 9,000,000, or 10,000,000 spermine molecules. In some embodiments, the spermine molecules may be non-covalently associated with the microbubble surface. For example, the spermine molecules may be associated with the microbubble surface through electrostatic interactions, such as an electrostatic interaction between the positive charges of one or more amino groups on the spermine to negative charges on the microbubble surface (e.g., negatively charged phosphate groups on the heads of phospholipids). In some embodiments, functionalized spermine molecules may be non-covalently associated with functional groups on the microbubble surface through non-covalent binding interactions, such as receptor-ligand binding, antibody-antigen binding, streptavidin-biotin binding, etc. In some embodiments, the spermine molecules may be covalently bound to the microbubble surface through conjugation chemistries known in the art, including those described elsewhere herein.

In some embodiments, one or more of the primary amines in the spermine molecule may be used to covalently couple the spermine molecule to a surfactant molecule in the surfactant shell of the microbubble (via a reactive functional group) or to an interlinking polymer configured to link the spermine molecule to the microbubble. In some embodiments, the primary amine may be covalently coupled with an isothiocyanate, an isocyanate, an acyl azide, an NHS ester, a sulfonyl chloride, an aldehyde, a ketone, a glyoxal, an epoxide, an oxirane, a carbonate, an arylating agent, an imdoester, a carbodiimide, an anhydride, a fluorophenyl ester, a hydroxymethyl phosphine derivative, or a guanadino group presented on the microbubble surface or on the interlinking polymer. For example, in some embodiments, the primary amine may be conjugated to a ketone, an aldehyde, or a glyoxal to form a Schiff base, which may be chemically stabilized by reduction (e.g., with borohydride or cyanoborohydride). For instance, one or more of the primary amines of a spermine molecule may be reacted with the aldehydes in oxidized dextran, as described elsewhere herein, or other oxidized polysaccharides.

### Interlinking Polymers

In some embodiments, at least some or all of the spermine molecules are associated with the microbubble via an interlinking polymer. In some embodiments, the spermine molecules may be conjugated to the interlinking polymer and then the interlinking polymer may be linked (e.g., covalently coupled) to the microbubble. In some embodiments, the microbubble may be first decorated with (e.g., covalently coupled to) the interlinking polymer and then the spermine molecules may be conjugated to the interlinking polymer. In some embodiments, the interlinking polymer may be joined to the spermine molecules and the microbubble in substantially simultaneous fashion, such as by mixing solutions comprising the spermine molecules, the microbubble composition, and the interlinking polymer together at one time.

In some embodiments, the interlinking polymer may be conjugated to at least two spermine molecules and at least one of the spermine molecules may be used to link the interlinking polymer to the microbubble. For instance, one of the primary amines on a spermine may be covalently linked to the interlinking polymer and the other primary amine may be linked to the microbubble. The two linking primary amines may be coupled to the interlinking polymer and the microbubble via the same type of conjugations or via different (e.g., biorthogonal) conjugations. The spermine molecules may be conjugated to either the interlinking polymer of the microbubble by any of the conjugation chemistries or non-covalent interactions described elsewhere herein. In some embodiments, a portion of the free primary amines on a spermine molecule may be thiolated (e.g., via 2-iminothiolane) for conjugation to the microbubble surface. The thiols on the spermine molecules may be conjugated to thiol-reactive groups such as maleimides exposed on the microbubble surface. In some embodiments, approximately 1 out of every 5, 6, 7, 8, 9, 10, 15, 20, 25, or 50 free primary amino groups, and/or approximately 5%, 10%, 15%, 20%, or 25% of the free primary amino groups within a spermine molecule may be functionalized for binding to the microbubble.

When a spermine molecule is used to link an interlinking polymer to the microbubble it is designed to indirectly link one or more additional spermines that are also linked to the interlinking polymer to the microbubble. In some embodiments, a plurality of spermine molecules are indirectly linked to the microbubble via an interlinking polymer. It is to be understood that reference to a plurality of spermine molecules being indirectly linked to the microbubble includes embodiments in which some of the spermine molecules are directly coupled to the microbubble, e.g., embodiments in which one or more of the spermine molecules are used as crosslinkers to join the interlinking polymer to the microbubble. In some embodiments, each interlinking polymer, such as a dextran (e.g., 40 kDa dextran), is linked to at least 1, 2, 3, 4, 5, 6, 7, 8, 9, 10, 15, 20, 25, 30, 35, 40, 45, 50, 60, 70, 80, 90, or 100 spermines. In some embodiments, each interlinking polymer, such as a dextran (e.g., 40 kDa dextran), is linked to 1-10, 10-50, 50-100, 10-20, 20-30, 30-40, 40-50, 50-60, 60-70, 70-80, 80-90, or 90-100 spermines. In some embodiments, at least 1, 2, 3, 4, 5, 6, 7, 8, 9, 10, 15, 20, 25, 30, 35, 45, 50, 60, 70, 80, 90, or 100 of the linked spermine molecules and/or at least 70%, 75% 80%, 85%, 90%, 95%, 99%, or 100% of the linked spermines molecules are “free” spermines. As used herein, a free spermine is a spermine in which at least one of its primary amines is not covalently reacted to any other moiety and is “free” to interact with a negatively charged payload, as described elsewhere herein. A free spermine may be conjugated to an interlinking polymer of microbubble by its other primary amino group or, for example, by a secondary amine. A polyspermine comprising at least one free primary amino group on each spermine is considered to comprise free spermines. A spermine molecule which crosslinks an interlinking polymer to a microbubble or which crosslinks together two interlinking polymers is not free.

In some embodiments, one or more of the spermines may be linked to an interlinking polymer via a biodegradable linker, such as those described elsewhere herein. In some embodiments, one or more of the interlinking polymers may be linked to microbubbles via a biodegradable linker, such as those described elsewhere herein.

The interlinking polymer may be a scaffold polymer in which a plurality of free spermine molecules are associated with each interlinking polymer. Scaffold polymers may be used to increase the loading capacity of spermine in spermine-decorated microbubble compositions by allowing the number of free spermines associated with each microbubble to be greater than the number of links (e.g., covalent bonds) between the microbubble and the interlinking polymers. In some embodiments, there are at least 2, 3, 4, 5, 6, 7, 8, 9, 10, 15, 20, 30, 35, 40, 45, 50, 60, 70, 80, 90, or 100 number of free spermine molecules for every covalent bond to the microbubble.

In some embodiments, the interlinking polymer comprises a linear (non-branched) polymer. One or more spermines may be conjugated to each interlinking polymer. Depending on the interlinking polymer and the conjugation chemistries used, spermine molecules may be conjugated to a terminal end of the polymer chain and/or to reactive groups within in the chain backbone. In some embodiments, the interlinking polymer comprises a branched polymer in which one or more branch chains extend from a linear polymer chain backbone. The branches may be linear or may themselves be branched. In some embodiments, spermine molecules are associated with the linear polymer chain backbone. In some embodiments, spermine molecules are associated with one or more polymer branches. In some embodiments, spermine molecules are associated with multiple branches of a branched interlinking polymer. In some implementations of the methods described herein, use of branched scaffold polymers may result in spermine-decorated polymer molecules with smaller hydrodynamic radii than spermine-decorated linear polymers of similar molecular weights. Branched polymers may increase the spermine carrying capacity of the microbubble relative to linear polymers of similar molecular weight. In some implementations of the methods described herein, use of branched scaffold polymers may result in spermine-decorated polymer molecules with higher spermine/payload densities than spermine-decorated linear polymers of similar hydrodynamic radii. Reducing the hydrodynamic radius of a spermine-decorated polymer and/or increasing the spermine/payload density of a spermine-decorated polymer of a given radius may increase the cellular uptake of payload in sonoporated cells in which the established transient pores have limited dimensions.

The interlinking polymer may be any polymer used in the art for forming drug delivery vehicles (e.g., polyplexes or nanoparticles). The interlinking polymer may be a biopolymer such as a polysaccharide (e.g., a glucan) or an extracellular matrix polymer or component thereof (e.g., hyaluronic acid, collagen, elastin, fibronectin, laminin, or proteoglycans such as heparan sulfate, chondroitin sulfate, keratin sulfate). In some embodiments the interlinking polymer may comprise dextran. In some embodiments, the interlinking polymer may comprise linear PEG. In some embodiments, the interlinking polymer may comprise branched PEG (e.g., 4-arm or 8-arm star-shaped PEG). In some embodiments, the interlinking may comprise one or more of poly(glycolic acid), poly(lactic acid), poly(lactic-co-glycolic) acid, polyhydroxybutyrate, polyhydroxyvalerate, polycaprolactone, polyanhydrides, polycyanoacrylate, poly(ortho ester), polyphosphazenes or copolymers thereof. The interlinking polymer may be selected for its spermine-loading capacity. The interlinking polymer may be selected for its biocompatibility (e.g., low toxicity). In some embodiments, the microbubble composition comprises multiple types of interlinking polymers. In some embodiments, the interlinking polymers comprise both linear and branched polymers.

The interlinking polymer may be dextran. Dextran, as used herein, refers to D-glucans that contain a substantial number of (1→6)-linked-α-D-glucopyranopsyl residues. The great majority of dextrans are produced by bacteria growing on sucrose as a substrate. Naturally occurring dextran is a complex branched glucan derived from the condensation of glucose. Dextran chains are of varying lengths (from 3 to 2000 kDa). The polymer main chain consists of α-1,6 glycosidic linkages between glucose monomers, with branches from α-1,3 linkages, as depicted in FIG. 3. Branches may have lengths of as little as two or three glucose units to greater than 50 glucose units, with lower molecular weight dextrans exhibiting less branching and narrower molecular weight distributions than higher molecular weight dextrans. Dextrans with molecular weight greater than 10,000 glucose units behave as if they are highly branched. Native dextran has been found to have a molecular weight (MW) in the range of 9 million to 500 million glucose units. As the molecular weight increases, dextran molecules attain greater symmetry. Dextrans with molecular weights of 2,000 to 10,000 glucose units tend to exhibit the properties of an expandable coil. At molecular weights below 2,000 glucose units, dextran tends to be more rod-like. Linear forms of dextran (lacking any branching) can be chemically synthesized. Decorating microbubbles with dextran may improve the anti-flocculation/colloidal stability, anti-immunogenicity, hydrophilicity, biocompatibility, and/or in vivo circulation time/bioavailability of the microbubbles. Dextran is a relatively non-toxic polymer suitable for in vivo applications, particularly as compared to PEI.

In some embodiments, the dextran may comprise an average molecular weight (across the microbubble composition) of approximately 5 kDa, 10 kDa, 15 kDa, 20 kDa, 25 kDa, 30 kDa, 35 kDa, 40 kDa, 45 kDa, 50 kDa, 60 kDa, 70 kDa, 80 kDa, 90 kDa, 100 kDa, 150 kDa, 200 kDa, 500 kDa, 1 MDa, 1.5 MDa, or 2 MDa. In some embodiments, the dextran may comprise an average molecular weight of at least approximately 5 kDa, 10 kDa, 15 kDa, 20 kDa, 25 kDa, 30 kDa, 35 kDa, 40 kDa, 45 kDa, 50 kDa, 60 kDa, 70 kDa, 80 kDa, 90 kDa, 100 kDa, 150 kDa, 200 kDa, 500 kDa, 1 MDa. In some embodiments, the dextran may comprise an average molecular weight no greater than approximately 50 kDa, 100 kDa, 150 kDa, 200 kDa, 500 kDa, 1 MDa, 1.5 MDa, or 2 MDa. In some embodiments, the dextran may comprise an average molecular weight of approximately 5-100 kDa, 10-80 kDa, 15-70 kDa, 20-65 kDa, kDa, 30-55 kDa, 35-50 kDa, 40-45 kDa, or 35-45 kDa, 35-40 kDa, or 20-50 kDa. In various embodiments, the interlinking polymers may be dialyzed at one or more molecular weight cutoffs to remove lower molecular weight species from the composition. The polymers may be dialyzed before coupling to spermine molecules, after coupling to spermine molecules, or at both times.

Decoration of dextran with spermine may increase the effective molecular weight of the polymer by approximately 1-5%, 5-10%, 10-15%, 15-20%, 20-25%, 10-20%, 10-30%, 20-30%, 10-50%, or more. In some embodiments, approximately 1 out of every 2, 3, 4, 5, 6, 7, 8, 9, 10, 10-20, 20-30, 30-40, 40-50, 10-25, 25-50, or 10-50 glucose monomers in a dextran molecule is decorated with spermine. However, aminolysis resulting from the spermine conjugation may reduce the molecular weight of the polymer by 1-5%, 5-10%, 10-15%, 15-20%, 20-25%, 10-20%, 10-30%, 20-30%, 10-50%, 50-75%, 50-60%, 70%-80%, 50-75%, or more. For instance, in some embodiments, 40 kDa dextran may be reduced to approximately 10 kDa after conjugation with spermine.

In some embodiments, each microbubble in the microbubble composition is ultimately decorated with about 1×10⁻¹⁵-1×10⁻¹³, 1×10⁻¹⁵-1×10⁻¹⁴, or 1×10⁻¹⁴-1×10⁻¹³ g of interlinking polymer, such as spermine-decorated dextran, per microbubble. For example, in some embodiments, each microbubble is decorated with at least about 1.0×10⁻¹⁴, 1.1×10⁻¹⁴, 1.2×10⁻¹⁴, 1.3×10⁻¹⁴, 1.4×10⁻¹⁴, 1.5×10⁻¹⁴, 1.6×10⁻¹⁴, 1.7×10⁻¹⁴, 1.8×10⁻¹⁴, 1.9×10⁻¹⁴, 2.0×10⁻14, 3.0×10⁻¹⁴, 4.0×10⁻¹⁴, 5.0×10⁻¹⁴, 6.0×10⁻¹⁴, 7.0×10⁻¹⁴, 8.0×10⁻¹⁴, or 9.0×10⁻¹⁴ g/microbubble. In some embodiments, each microbubble in the microbubble composition is ultimately decorated with at least about 25,000, 50,000, 75,000, 100,000, 150,000, 200,000, 300,000, 400,000, 500,000, 600,000, 700,000, 800,000, 900,000, or 1,000,000 interlinking polymers, such as spermine-decorated dextrans.

### Microbubble Targeting

The microbubbles may additionally be decorated with targeting molecules, which bind to specific cell types or other biological structures. In some embodiments, the targeting molecule may be a protein or other biomolecule (e.g., a ligand for a cell surface receptor). In some embodiments, the targeting molecule may be a biopolymer or component thereof, such as an extracellular matrix polymer (e.g., hyaluronic acid, collagen, elastin, fibronectin, laminin, or proteoglycans such as heparan sulfate, chondroitin sulfate, keratin sulfate). In some embodiments, the targeting molecule is a monoclonal or polyclonal antibody, including antibody fragments or peptides derived from/modeled after antibodies with antigen-binding properties. For instance, the antibody may be a Fab fragment, an F(ab′)2 fragment, an Fab′ fragment, an Fv fragment, an scFv, a di-scFv, an sdAb, a recombinant IgG, a peptide comprising one or more complementary determining regions (CDRs), or any other antibody fragment or biomolecule with antigen binding properties well known in the art. The antibody may be specific for an antigen expressed on the cell surface of the targeted cell type (e.g., a cell surface receptor). In some embodiments, the microbubbles may be configured to target cancerous/tumor cells. In some embodiments, the microbubbles may be configured to target immune cells (e.g., T-cells, B, cells, neutrophils, eosinophils, basophils, mast cells, monocytes, macrophages, dendritic cells, natural killer cells, etc.). The targeting molecules may be covalently coupled to the external surface of the surfactant shell of the microbubble.

The targeting molecules may be associated with the external surface of the microbubble in the same manner as the spermine molecules. For instance, the targeting molecules may be covalently linked to functional groups on the external surface of the surfactant shell in the same manner as the spermine molecules (e.g., the microbubble may be linked to the targeting molecule or to an interlinking polymer). The targeting molecules may be linked to the same functional groups on the microbubble as the spermine molecules (e.g., using the same pair of reactive groups or a third reactive group that reacts with the same reactive group as does the functionalized spermine molecules) or may be linked to different functional groups. In some embodiments, the targeting molecules and spermine molecules may be linked to the microbubbles using biorthogonal reactions in which there is no cross-reactivity between the targeting molecule and the spermine molecule, between the targeting molecule and the functional group on the microbubble surface configured to bind the spermine molecule, or between the spermine molecule and the functional group on the microbubble surface configured to bind the targeting molecule. The targeting molecules may be coupled to the microbubbles by incubating the targeting molecules with the microbubble composition (e.g., mixing a solution comprising targeting molecules with a solution comprising a microbubble composition). The targeting molecules may be coupled to the microbubbles prior to, simultaneously with, or subsequent to coupling the spermine molecules to the microbubbles. In some embodiments, the targeting molecules and the spermine molecules may be coupled to the same interlinking polymer such that the interlinking polymer links both the spermine molecules and targeting molecules to the microbubble. In some embodiments, each microbubble in the microbubble composition is decorated with at least about 5,000, 10,000, 15,000, 20,000 25,000, 50,000, 75,000, 100,000, 200,000, 300,000, 400,000, 500,000, 600,000, 700,000, 800,000, 900,000, or 1,000,000 targeting molecules.

### Microbubble Payloads

The microbubbles described herein may be used to bind payloads for drug delivery, such as through sonoporation as described elsewhere herein. In various embodiments, the spermine molecules of the spermine-decorated microbubbles serves as a drug delivery vehicle (e.g., transfection agent) for the payload by binding to the payload. In some embodiments, the payload comprises a protein, such as a protein therapeutic. Protein therapeutics may comprise, for example, antibody-based drugs, anticoagulants, blood factors, bone morphogenetic proteins, engineered protein scaffolds, enzymes, Fc fusion proteins, growth factors, hormones, interferons, interleukins, thrombolytics, etc. In some embodiments, the payload comprises a nucleic acid. The nucleic acid may be, for example, a plasmid, an siRNA, or a Dicer-substrate siRNA (DsiRNA). In some embodiments, the nucleic acid may be a DNA, which may be delivered, for instance, as part of a gene therapy. Gene therapy may target either somatic cells or germline cells and can be used to treat conditions such as immunodeficiencies, haemophilia, thalassaemia, and cystic fibrosis. Gene therapy may be performed ex vivo, in some applications, on hematopoietic stem cells. Examples of genes that may be delivered include genes for Factor IX, human fibroblast growth factor-4, ND4 protein, INF alpha-2b, adrenoleukodystrophy protein (ABCD1 gene), N-sulfoglucosamine sulfohydrolase (SGSH), connexin43, REP1, arylsulfatase A, 5T4 oncofetal antigen, thymidine kinase, AC6, cytosine deaminase, Factor VIII, hepatocyte growth factor (e.g., HGF728, HGF723), SMN protein, β-globin, etc. In some embodiments, the nucleic acid may be an RNA. In various embodiments, the nucleic acid may be modified. For example, the nucleic acid may comprise non-naturally occurring nucleotides, including molecules comprising both deoxyribonucleotides and deoxynucleotides.

The payload may bind to the microbubbles via electrostatic interactions between positive charges on the microbubble surface (e.g., the positively charged primary and/or secondary amino groups of spermine molecules) and negative charges exposed on the payload molecules, such as negatively charged phosphate groups on the sugar phosphate backbone of the nucleic acid. The microbubble compositions described herein may be loaded with nucleic acids according to the ratio of the number of positively-chargeable amine groups (N) decorating the microbubbles of the microbubble composition to the number of negatively-charged nucleic acid phosphate groups (P) within the payload composition. In some embodiments, the microbubble composition may be loaded at N:P ratios of approximately 1:1, 1:2, 1:3, 1:4, 1:5, 1:6, 1:7, 1:8, 1:9, 1:10, 1:11, 1:12, 1:13, 1:14, 1:15, 1:16, 1:17, 1:18, 1:19: or 1:20. In some embodiments, the microbubble composition may be loaded at N:P ratios of at least about 1:5, 1:10, 1:15, or 1:20. In some embodiments, the microbubble composition may be prepared at concentrations of approximately 1×10⁻⁷-1×10⁻¹⁰, 1×10⁻⁷-1×10⁻⁹, 1×10⁻⁷-1×10⁻⁸, 1×10⁻⁸-1×10⁻¹⁰, or 1×10⁻⁸-1×10⁻⁹ μg of payload (e.g., pDNA) per microbubble. The microbubble composition may, for example, be prepared at concentrations of at least about 1×10⁻⁸, 2×10⁻⁸, 3×10⁻⁸, 4×10⁻⁸, 5×10⁻⁸, 6×10⁻⁸, 7×10⁻⁸, 8×10⁻⁸, 9×10⁻⁸ μg/microbubble.

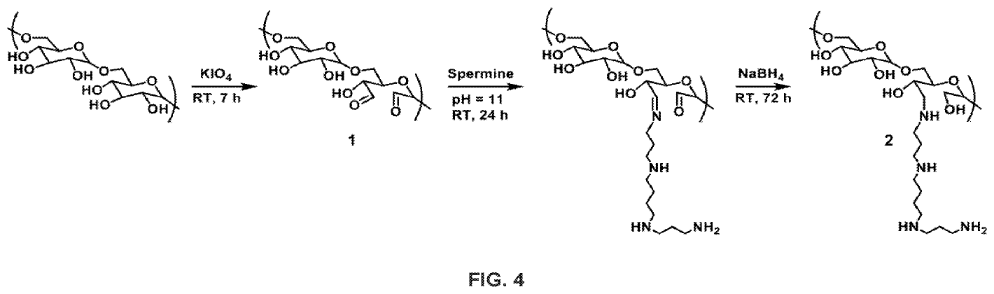

The amount of nucleic acid that a microbubble composition is ultimately able to carry depends in part on the amount of positive charges available (e.g., the surface density of the amino groups on the microbubble surface) as well as the ability of the positively charged polymer to stably bind the nucleic acid. The binding stability of a microbubble composition may generally be reduced at higher loading capacities. Microbubble compositions of the present disclosure generally demonstrate more stable loading than microbubble compositions known in the art and may effectively achieve higher loading capacities than microbubble compositions with higher numbers and/or densities of positive charges. For instance, PEI provides a primary amine density of approximately 8.2 mol per mg of polymer. A spermine-dextran in which every other glucose unit is attached to a spermine as depicted in FIG. 4 would provide a density of approximately 2.0 mol per mg of polymer. Yet, the results disclosed herein demonstrate that the spermine-decorated microbubbles described herein are able to load significantly more DNA than PEI-conjugated microbubbles, even when far fewer than 1 out of every 2 glucose units of the dextran is effectively conjugated to a spermine. In some embodiments, the microbubble compositions disclosed herein may exhibit a loading capacity of at least about 5,000, 10,000, 15,000, 20,000, 25,000, 30,000, or 35,000 nucleic acid molecules per microbubble. In some embodiments, the microbubble compositions disclosed herein may exhibit a loading capacity of at least about 0.010, 0.015, 0.020, 0.025, 0.030, 0.035, 0.045, 0.050, or 0.100 μg/μm 2 of nucleic acid relative to the calculated surface area of the microbubble composition. In some embodiments, the microbubble compositions disclosed herein may exhibit a loading capacity of at least about one nucleic acid every 10, 25, 50, 75, 100, 125, 150, 275, 200, 250, or 500 spermines.

### Methods of Making Microbubble Compositions

Microbubble compositions comprising spermine-decorated microbubbles may generally be prepared by combining solutions comprising the aforementioned components, which comprise microbubbles, spermine molecules, optionally interlinking polymers such as dextran, optionally payload molecules, such as nucleic acids, and optionally targeting molecules, such as antibodies. Each solution may be prepared as described elsewhere herein. Different solutions may be combined according to the methods described elsewhere herein. The simple preparation described herein provides advantages of convenience and speed over microbubbles loaded via layer-by-layer deposition of payload.

In various embodiments, the order of combining two solutions and/or the speed at which the solutions are combined may influence the resulting composition. For instance, a solution comprising a first component may be added slowly or gradually to a solution comprising a second component if it is desired that the first component react with the second component under conditions where the second component is in excess of the first component. Adding the first component slowly or gradually (e.g., drop wise or under a timed syringe pump) to the second component may promote the saturation of the first component with the second component in contexts where one molecule of the first component is able to react with multiple molecules of the second component. Slowly or gradually may be understood to be at a speed or rate which promotes substantial saturation and may depend on the kinetics of the particular reaction. The final molar ratios of the resulting mixture may also affect the degree of saturation. A mixture having a final molar ratio of the number of reactive groups within the second solution being in substantial excess to the number of corresponding reactive groups within the first solution may promote the saturation of the first component with the second component. In some embodiments, the number of reactive groups within the second solution may be present in a final molar ratio of at least about 2:1, 3:1, 4:1, 5:1, 6:1, 7:1, 8:1, 9:1, 10:1, 15:1, 20:1, 25:1, 75:1 or 100:1 relative to the number of reactive groups within the first solution in order to promote saturation of the first component with the second component.

In some embodiments, a solution comprising an interlinking polymer, such as dextran (e.g., oxidized dextran), is slowly or gradually added to a solution comprising the spermine molecules such that each interlinking polymer is substantially saturated with spermine molecules. Slowly or gradually adding the polymer solution to the spermine solution minimizes the amount of crosslinking between polymers by the spermine. In some embodiments, substantially no polymers are crosslinked at this stage. For instance, less than 1%, 2%, 3%, 4%, 5%, 6%, 7%, 8%, 9%, 10%, or 15% of the polymers are crosslinked to another polymer. In embodiments, where the primary amines of the spermine are covalently coupled to the interlinking polymer, promoting saturation and reducing the number of spermine crosslinkers between molecules of polymer increases the number of free spermines and free primary amino groups available to partake in subsequent reactions or to bind with payload. In some embodiments, the solution of spermine-decorated polymer may be dialyzed to remove unreacted spermine prior to subsequent preparation steps.

In other embodiments, a solution comprising spermine molecules is slowly or gradually added to a solution comprising an interlinking polymer, such as dextran. Slowly or gradually adding the solution comprising spermine molecules to the polymer solution may promote increased crosslinking between polymers. For instance, at least than 1%, 2%, 3%, 4%, 5%, 6%, 7%, 8%, 9%, 10%, 15%, 20%, 25%, 30%, 40%, or 50% of the polymer molecules may be crosslinked to at least one additional polymer molecule. Crosslinking polymers may increase the effective molecular weight of the spermine-polymer complex. Crosslinking polymers may effectively increase the relative amount of branching in the polymers that decorate the microbubble surface.

In some embodiments, the spermine molecules are functionalized for association (e.g., covalent coupling) with the microbubbles after being linked to the interlinking polymer, such as dextran. The spermine-decorated polymer may be functionalized under conditions, such as molar ratios, that prevent saturation of the primary amino groups with functional groups for reacting with the microbubble surface. In some embodiments, approximately 1 out of every 5, 6, 7, 8, 9, 10, 15, 20, 25, or 50 free amino groups, and/or approximately 5%, 10%, 15%, 20%, or 25% of the free primary amino groups within a spermine molecule may be functionalized for binding to the microbubble, such as with a thiol group.

In some embodiments, a solution comprising microbubbles is slowly or gradually added to a solution comprising spermine molecules or a spermine-decorated polymer, such as spermine-decorated dextran, such that each microbubble is substantially saturated with spermine molecules/spermine decorated polymer. Slowly or gradually adding the microbubble solution to the spermine comprising solution may minimize the amount of crosslinking between microbubbles by the spermine or spermine-modified polymer. In some embodiments, substantially no microbubbles are crosslinked at this stage. For instances, less than 1%, 2%, 3%, 4%, 5%, 6%, 7%, 8%, 9%, 10%, or 15% of the microbubbles are crosslinked to another microbubble. Promoting saturation of the microbubbles and reducing the number of crosslinks between microbubbles may increase the number of free spermines and free primary amino groups available to partake in subsequent reactions or to bind with payload. In some embodiments, the solution of spermine-decorated microbubbles may be dialyzed or washed to remove unbound spermine and/or polymer prior to subsequent preparation steps.

In other embodiments, a solution comprising spermine molecules or a spermine-decorated polymer is slowly or gradually added to a solution comprising microbubbles. Slowly or gradually adding the solution comprising spermine to the microbubble solution may promote increased crosslinking between microbubbles. For instance, at least than 1%, 2%, 3%, 4%, 5%, 6%, 7%, 8%, 9%, 10%, 15%, 20%, 25%, 30%, 40%, or 50% of the microbubbles may be crosslinked to at least one additional microbubble. In some embodiments, the average microbubble may be crosslinked to 1, 2, 3, 4, 5, 6, 7, 8, 9, or 10 additional microbubbles. Crosslinking microbubbles may increase the effective size and surface area of an individual particle, which may comprise one microbubble or multiple microbubbles crosslinked together, enhancing the payload loading capacity per particle. In some implementations of the methods described herein, increasing the payload loading capacity per particle may increase the efficiency of payload delivery to cells, such as in some sonoporation applications, described elsewhere herein. In some embodiments, the effective diameter of the microbubble particle may be approximately 1 μm, 2 μm, 3 μm, 4 μm, 5 μm, 6 μm, 7 μm, 8 μm, 9 μm, 10 μm, or greater than 10 μm.

In some embodiments, a solution comprising spermine-decorated microbubbles is slowly or gradually added to a solution comprising a payload, such as a nucleic acid (e.g., plasmid DNA or pDNA), such that each spermine-decorated microbubble is substantially saturated with payload molecules. Slowly or gradually adding the spermine-decorated microbubble solution to the payload solution minimizes the amount of crosslinking between microbubbles by the payload molecules. In some embodiments, substantially no microbubbles or particles comprising spermine- or polymer-crosslinked microbubbles are crosslinked (or further crosslinked) at this stage. For instances, less than 1%, 2%, 3%, 4%, 5%, 6%, 7%, 8%, 9%, 10%, or 15% of the microbubbles are crosslinked to another microbubble. Promoting saturation of the microbubbles or microbubble particles with payload may increase the efficiency of payload delivery to cells, such as in some sonoporation applications, described elsewhere herein.

In other embodiments, a payload solution is slowly or gradually added to a solution comprising spermine-decorated microbubbles. Slowly or gradually adding the payload solution to the spermine-decorated microbubble solution may promote increased crosslinking between microbubbles. For instance, at least than 1%, 2%, 3%, 4%, 5%, 6%, 7%, 8%, 9%, 10%, 15%, 20%, 25%, 30%, 40%, or 50% of the microbubbles may be crosslinked to at least one additional microbubble. In some embodiments, the average microbubble may be crosslinked to 1, 2, 3, 4, 5, 6, 7, 8, 9, or 10 additional microbubbles. Crosslinking microbubbles may increase the effective size and surface area of an individual particle, which may comprise one microbubble or multiple microbubbles crosslinked together, enhancing the payload loading capacity per particle. In some implementations of the methods described herein, increasing the payload loading capacity per particle may increase the efficiency of payload delivery to cells, such as in some sonoporation applications, described elsewhere herein. In some embodiments, the effective diameter of the microbubble particle may be approximately 1 μm, 2 μm, 3 μm, 4 μm, 5 μm, 6 μm, 7 μm, 8 μm, 9 μm, 10 μm, or greater than 10 μm.

In various embodiments, the solutions comprising spermine, polymer, spermine-decorated polymer, microbubbles, spermine-decorated microbubbles, and/or payload may be combined together through standard means known in the art. In some embodiments, the solutions may be combined slowly or gradually as described elsewhere herein, e.g., using a syringe pump or titration flask. Solutions may be combined over periods of approximately 5 min, 10 min, 15 min, 30 min 45 min, 1 h, 2 h, 3 h, 4 h, 5 h, 6 h, 7 h, 8 h, 9 h, 10 h, 12 h, 18 h, or 24 h or as otherwise needed to allow the reactions to proceed to substantial completion. Solutions may be mixed during and/or after combination of solutions through standard means known in the art, including, rotation, stirring, shaking, etc. Solutions may be mixed over periods of approximately 15 min, 30 min 45 min, 1 h, 2 h, 3 h, 4 h, 5 h, 6 h, 7 h, 8 h, 9 h, 10 h, 12 h, 18 h, or 24 h after combining. In some embodiments, a first component is in dry form (e.g., lyophilized) and mixed with a second component in solution, by dissolving the first component in the second component. In some embodiments, the dry component can be a component which is slowly or gradually added to a solution. At various steps, centrifugation may be used to wash or otherwise separate microbubbles from other components in a reaction solution. The microbubbles may be collected in a buoyant supernatant.

### Methods of Using Microbubble Compositions for Drug Delivery

### Sonoporation

In one aspect of the disclosure are provided methods for delivering payloads to one or more cells using the microbubbles described herein with sonoporation techniques. Sonoporation uses sound, typically at ultrasonic frequencies, for increasing the permeability of the cell plasma membrane in order to facilitate the delivery of a payload across the membrane and into the cell. Sonoporation may be particularly beneficial for delivering payloads across the blood-brain barrier or blood-tumor barrier. For in vivo applications, sonoporation may be an advantageous means of drug delivery as ultrasound can penetrate deep into the subject's tissue in a non-invasive manner as well as provide spatially and temporally targeted delivery with minimal or no side effects to non-targeted tissue. Microbubbles can function as nuclei for acoustic cavitation in ultrasound-mediated drug delivery, effectively scattering ultrasound waves due to the high compressibility of the microbubbles. Sonoporation is believed to induce transient increases in cell permeability via the formation of transient pores in the cell plasma membrane. Without being bound by theory, collapsing microbubbles may produce local shock waves, water jets, and shear forces that are able to permeabilize nearby cell membranes. Sonoporation may allow the direct delivery (i.e. outside the endosomal transport pathway) of therapeutics, such as nucleic acids, into a cell's cytosol.

The intensity of the ultrasound waves and the composition of the microbubble shell may influence the effectiveness of the sonoporation. Microbubble shells should be stiff enough to withstand small pressure perturbations but elastic enough to oscillate in response to the ultrasound waves. Lower degrees of polydispersity in microbubble size may be desirable so that larger proportions of a microbubble composition are sensitive to the same amplitudes of ultrasound. Without being limited by theory, microbubbles subjected to low-intensity ultrasound may oscillate stably around a resonant diameter, termed stable cavitation. Stable cavitation generates local shear forces and acoustic microstreaming. At higher pressure amplitudes, microbubbles tend to undergo large size variations which cause them implode in an event termed inertial cavitation, the collapse resulting in water jetting, shock waves and other inertial phenomena. Both stable and transient microbubble cavitations may induce cell membrane permeabilization. Sonoporation is believed to provide a pathway to intracellular drug delivery independent of endocytosis. In some embodiments, ultrasound triggering of sonoporation with microbubbles may be performed at frequencies between about 0.1 MHz and about 10 MHz, between about 0.5 MHz and about 5 MHz, or between about 1 MHz and about 3 MHz. In some embodiments, ultrasound triggering may be performed at intensities between about 100 mW/cm² and about 1 kW/cm², between about 100 mW/cm² and about 1 W/cm², between about 300 mW/cm² and about 1 W/cm², between about 500 mW/cm² and about 1 W/cm², between about 700 mW/cm² and about 1 W/cm², between about 1 W/cm² and about 500 W/cm², between about 1 W/cm² and about 100 W/cm², between about 1 W/cm² and about 100 W/cm², between about 1 W/cm² and about 50 W/cm², between about 1 W/cm² and about 20 W/cm², between about 1 W/cm² and about 10 W/cm², between about 1 W/cm² and about 5 W/cm², or between about 1 W/cm² and about 2 W/cm². In some embodiments, the ultrasound triggering may be performed at a maximal intensity permitted by a regulatory agency (e.g., the FDA), such as, for example, approximately 720 mW/cm² (for diagnostic ultrasound). In some embodiments, the ultrasound triggering may be performed at a duty cycle between about 10% and 100%, between about 20% and about 90%, between about 30% and about 80%, between about 40% and about 70%, or between about 50% and about 60%. In some embodiments, the duty cycle is about 50%. In some embodiments, the mechanical index (MI) of the ultrasound may be between about 0.05 and about 5, between about 0.1 and about 5, between about 0.5 and about 5, between about 1 and about 5, between about 2 and about 5, between about 3 and about between about 4 and about 5. In some embodiments, the ultrasound triggering may be performed at a maximal mechanical index permitted by a regulatory agency (e.g., the FDA), such as, for example, approximately 1.9 (for diagnostic ultrasound). In some embodiments, the ultrasound triggering may be delivered for about 10 s-3 min, 10 s-2 min, 10 s-1 min, 10 s-50 s, 10 s-40 s, 10 s-30 s, 30 s-3 min, 30 s-2 min, 30 s-1 min, 30 s-50 s, 30 s-40 s, 1 min-3 min, or 1 min-2 min, 2 min-3 min.

### Drug Delivery Via Endocytosis

In some embodiments, delivery of the payload may occur and/or be induced without sonoporation, for example, by endocytosis (e.g., phagocytosis, pinocytosis, receptor-mediated endocytosis). In some embodiments, the targeting molecule and or another molecule decorating the spermine-decorated microbubbles may interact with a receptor that induces receptor-mediated endocytosis. In various embodiments, the spermine-decorated microbubbles may be incorporated into lysosomes, in which the pH is approximately 4.5-5.0. In various embodiments, the reduced pH within the lysosome may promote the cleavage of pH-sensitive biodegradable linkers (e.g., via the reduction of a disulfide bond), such as a cystamine bisacrylamide linker, described elsewhere herein. The degradation of biodegradable linkers between spermine molecules, between spermine molecules and interlinking polymers, and/or between interlinking polymers and the microbubble, may effectively free the spermine-associated payload and allow more effective delivery. For instance, disassociation of the payload from the microbubble may promote nuclear localization in some embodiments. The spermine-decorated microbubbles and payload may be prepared and/or combined with cells as described elsewhere herein.

### In Vitro Applications

In some embodiments, the drug delivery (and sonoporation where applicable) may be performed in vitro. The spermine-decorated microbubble composition may be combined with adherent cells or with cells in solution. When the microbubbles are added to cells in solution the sonoporation may be performed on the cells in solution or the cells may be subsequently allowed to adhere (may be plated) prior to sonoporation. Microbubbles may be combined with cells at a ratio of about 1:1-100:1, 5:1-50:1, 5:1-25:1, 10:1-50:1, 10:1-30:1, 10:1-25:1, 10:1-10:1-15:1, 15:1-20:1, or 15:1-25:1 microbubbles per cell. For example, in some embodiments the microbubbles and cells are combined at ratios of approximately 10:1, 15:1, or 20:1 microbubbles per cell. The spermine-decorated microbubble composition may be incubated for a period of time, e.g., 5 min, 10 min, 15 min, 20 min, 25 min, 30 min, 45 min, 1 h, etc., to allow the spermine-decorated microbubbles to sufficiently mix with and possibly target the cells (e.g., where targeting molecules are incorporated). In some embodiments, the microbubbles may be actively mixed (e.g., via rotation) with the cells, particularly when the microbubbles are combined with cells in solution. In embodiments in which the microbubbles are combined with adherent cells, the cell culture surface may be inverted for a portion or the entirety of the combination period to allow the buoyant microbubbles to better interact with the adherent cells. The cell/microbubble composition may be washed one or more times after mixing. In some embodiments the spermine-decorated microbubbles are pre-loaded with a payload (e.g., pDNA) prior to combining the microbubbles with the cells. In some embodiments, the payload is combined with the cells at substantially the same time as the microbubbles. For instance, the payload may be combined into the microbubble solution just prior to combination with the cells or substantially simultaneously with the cells. In some embodiments, the payload may be combined with cells during the incubation period allowing the microbubbles to mix with and/or target the cells. In some embodiments, the payload may be combined with the cells after the microbubbles have been allowed to mix with and/or target the cells. For instance, the payload may be combined with the cells after a washing step following the microbubble combination with the cells. In some embodiments in which the microbubbles are not pre-loaded with the payload, the payload may be incubated with the cells for a period of time, e.g., 5 min, 10 min, 15 min, 20 min, 25 min, 30 min, 45 min, 1 h, etc., prior to sonoporation to allow the payload to interact with the spermine on the spermine-decorated microbubbles. After the cells have been sufficiently combined with the microbubbles and payload, sonoporation may be performed as described elsewhere herein.

### In Vivo Applications

In some embodiments, the drug delivery (and sonoporation where applicable) may be performed in vivo on a subject A subject may include any organism capable of experiencing a beneficial effect from delivery of a payload. Examples of subjects include mammals, e.g., humans, dogs, cows, horses, pigs, sheep, goats, cats, mice, rabbits, rats, and transgenic non-human animals.

The microbubble and/or payload compositions (therapeutic compositions) described herein can be delivered in vivo by administration to an individual subject, typically by systemic administration (e.g., intravenous, intraperitoneal, intramuscular, subdermal, or intracranial infusion) or topical application. In some embodiments, the therapeutic composition may comprise a microbubble composition pre-loaded with the payload. In some embodiments, the therapeutic compositions may comprise separate microbubble compositions and payload compositions which are administered sequentially or substantially simultaneously. When administered sequentially, the payload composition may be administered to the subject prior to or following the administration of the microbubble composition.

In some embodiments, the compositions can be delivered to cells ex vivo, such as cells explanted from an individual subject (e.g., lymphocytes, bone marrow aspirates, tissue biopsy) or universal donor hematopoietic stem cells, followed by reimplantation of the cells into a subject, usually after selection for cells which have incorporated the payload. Ex vivo cell transfection for diagnostics, research, or for molecular therapy (e.g., via re-infusion of the transfected cells into the host organism) is well known to those of skill in the art. Various cell types suitable for ex vivo transfection are well known to those of skill in the art. In one embodiment, stem cells are used in ex vivo procedures for cell transfection and molecular therapy. Stem cells can be isolated for transduction and differentiation using known methods.

The therapeutic compositions can also be administered directly to an organism for transduction of cells in vivo. Administration is by any of the routes normally used for introducing a molecule into ultimate contact with blood or tissue cells including, but not limited to, injection, infusion, topical application and electroporation. Suitable methods of administering such therapeutic compositions are available and well known to those of skill in the art, and, although more than one route can be used to administer a particular composition, a particular route can often provide a more immediate and more effective reaction than another route.

The therapeutic compositions may be administered in the form of a pharmaceutical composition. Pharmaceutical compositions include preparations suitable for administration to mammals, e.g., humans. When the compounds of the present invention are administered as pharmaceuticals to mammals, e.g., humans, they can be given per se or as a pharmaceutical composition containing, for example, 0.1 to 99.5% (more preferably, 0.5 to 90%) of active ingredient in combination with a pharmaceutically acceptable carrier. Pharmaceutically acceptable carriers are known in the art and include a pharmaceutically acceptable material, composition or vehicle, suitable for administering compounds of the present disclosure to mammals. The carriers include liquid or solid filler, diluent, excipient, solvent or encapsulating material, involved in carrying or transporting the subject agent from one organ, or portion of the body, to another organ, or portion of the body. Each carrier should be “acceptable” in the sense of being compatible with the other ingredients of the formulation and not injurious to the patient. Some examples of materials which can serve as pharmaceutically acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate and magnesium stearate, as well as coloring agents, release agents, coating agents, sweetening, flavoring and perfuming agents, preservatives and antioxidants can also be present in the compositions.

Examples of pharmaceutically acceptable antioxidants include: water soluble antioxidants, such as ascorbic acid, cysteine hydrochloride, sodium bisulfate, sodium metabisulfite, sodium sulfite and the like; oil-soluble antioxidants, such as ascorbyl palmitate, butylated hydroxyanisole (BHA), butylated hydroxytoluene (BHT), lecithin, propyl gallate, α-tocopherol, and the like; and metal chelating agents, such as citric acid, ethylenediamine tetraacetic acid (EDTA), sorbitol, tartaric acid, phosphoric acid, and the like.

Pharmaceutically acceptable carriers are determined in part by the particular composition being administered, as well as by the particular method used to administer the composition. Accordingly, there is a wide variety of suitable formulations of pharmaceutical compositions available (see, e.g., Remington's Pharmaceutical Sciences, 17th ed., 1989).

In some embodiments, the therapeutic compositions, alone or in combination with other suitable components, can be made into aerosol formulations (i.e., they can be “nebulized”) to be administered via inhalation. Aerosol formulations can be placed into pressurized acceptable propellants, such as dichlorodifluoromethane, propane, nitrogen, and the like.

Formulations suitable for parenteral administration, such as, for example, by intravenous, intramuscular, intradermal, and subcutaneous routes, include aqueous and non-aqueous, isotonic sterile injection solutions, which can contain antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. The disclosed compositions can be administered, for example, by intravenous infusion, orally, topically, intraperitoneally, intravesically or intrathecally. The formulations of compounds can be presented in unit-dose or multi-dose sealed containers, such as ampules and vials. Injection solutions and suspensions can be prepared from sterile powders, granules, and tablets of the kind previously described.

For therapeutic applications, the dose of the therapeutic compositions administered to a subject, or to a cell which will be introduced into a subject, in the context of the present disclosure, should be sufficient to effect a beneficial therapeutic response in the subject over time. In addition, particular dosage regimens can be useful for determining phenotypic changes in an experimental setting, e.g., in functional genomics studies, and in cell or animal models. The dose will be determined by the efficacy and Kd of the particular compositions employed, the nuclear volume of the target cell, and the condition of the subject, as well as the body weight or surface area of the subject to be treated. The size of the dose also will be determined by the existence, nature, and extent of any adverse side-effects that accompany the administration of a particular compound or oligonucleotide agents in a particular subject. The dosages administered will vary from subject to subject; a “therapeutically effective dose” can be determined, for example, by monitoring symptoms or phenotypes, such as the size or growth rate, or the duration of the growth period of a tumor, tumor number, cancer cell number, viability, growth rate and the duration of the growth period of a cancer cell. In determining the effective amount of the therapeutic compositions to be administered in the treatment or prophylaxis of disease, the physician evaluates circulating plasma levels of the microbubbles and/or payload, potential toxicities, progression of the disease, and the production of anti-therapeutic antibodies. Administration can be accomplished via single or divided doses.

In various embodiments, the microbubble compositions and/or drug delivery methods described herein may be enhance the transfection efficiency of a nucleic acid payload (e.g., expression of a pDNA) by approximately 5%, 10%, 15%, 20%, 25%, 30%, 40%, 50%, 60%, 70%, 80%, 90%, 100%, or more, relative to a negative control.

### Kits Comprising Microbubble Compositions

Disclosed herein are also kits for preparing one or more of the compositions described elsewhere herein. The kits may provide one or more of the components in separate storage containers (e.g., vials, pouches, etc.) for use in applications such as the methods described herein. In some embodiments, the kit comprises a spermine-decorated microbubble composition as described elsewhere herein. The payload may or may not be included in the kit. In some embodiments, the spermine-decorated microbubble composition is preloaded with a payload. In some embodiments, the microbubble composition is not decorated with spermine, but the spermine molecules are provided in another composition (e.g., a spermine-decorated polymer composition) that can be combined with the microbubble composition. In some embodiments, one or more of the compositions are provided in solution. The solutions may be configured to be in liquid form at room temperature. In some embodiments, the solutions are configured to be frozen during storage. In some embodiments, the solutions are configured to be refrigerated during storage. The solutions may comprise stabilizing agents as generally known in the art. The storage containers may protect the compositions from light. In some embodiments, one or more of the compositions are provided in a dry or solid form. For instance, one or more solutions comprising the compositions may be put into dry form by lyophilizing or spray drying. In some embodiments, reagents (e.g., solutions) for reconstituting one or more dry compositions into liquid form are provided as part of the kit. In some implementations, the microbubble composition is suitable for reconstitution with an aqueous solution as well as a gas for reconstituting the gas cores of the microbubbles. The kit may comprise one or more of the agents necessary to combine any of the compositions, such as combining the spermine with the microbubbles or combining spermine-decorated microbubbles with payload. In some embodiments, the kit comprises one or more reagents or tools necessary for performing sonoporation.

As used herein, including in the following examples, when reference is made to “each” microbubble in a microbubble composition or to “each” polymer associated with a microbubble or within a composition, or an individual microbubble or polymer is otherwise characterized, it is to be understood that the characterizations are based on properties/measurements for bulk microbubble or polymer compositions with the assumption that the microbubble/polymer composition is substantially homogenous across the composition. Accordingly, average numbers (based on measured or estimated total numbers of microbubbles and/or total polymer number or mass) may be used to characterize “each” microbubble or polymer in the composition with the understanding that some variability is expected between each individual microbubble and/or polymer. Substantially each/every microbubble in a composition or each/every polymer associated with a microbubble may refer to, for instance, 80%, 90%, 95%, 96%, 97%, 98%, 99%, 99.1%, 99.2%, 99.3%, 99.4%, 99.5%, 99.6%, 99.7%, 99.8%, 99.9%, 99.99%, or 100% of the microbubbles/polymers (by mass or number) within the composition. Substantially each/every microbubble in a composition or each/every polymer associated with a microbubble may refer to an amount necessary to achieve any of the amounts of payload loading described elsewhere herein.

Any molecular weight averages disclosed herein may be either number-averaged or weight-averaged unless explicitly stated otherwise. Any average sizes disclosed herein (e.g., microbubble size) may be either number-averaged or size-averaged unless explicitly stated otherwise.

All measurements or values disclosed herein should be assumed to be approximate, unless explicitly stated otherwise. Measurements or values deemed to be “about” or “approximately” may be assessed by those skilled in the art in the context of the disclosure. For instance, unless indicated otherwise a measurement or value disclosed herein may have a deviation of no more than 20%, preferably no more than 10%, and more preferably no more than 5% without effectively departing from the subject matter described herein.

### EXAMPLES

### Example 1: Synthesis of Oxidized Dextran (O-Dex)

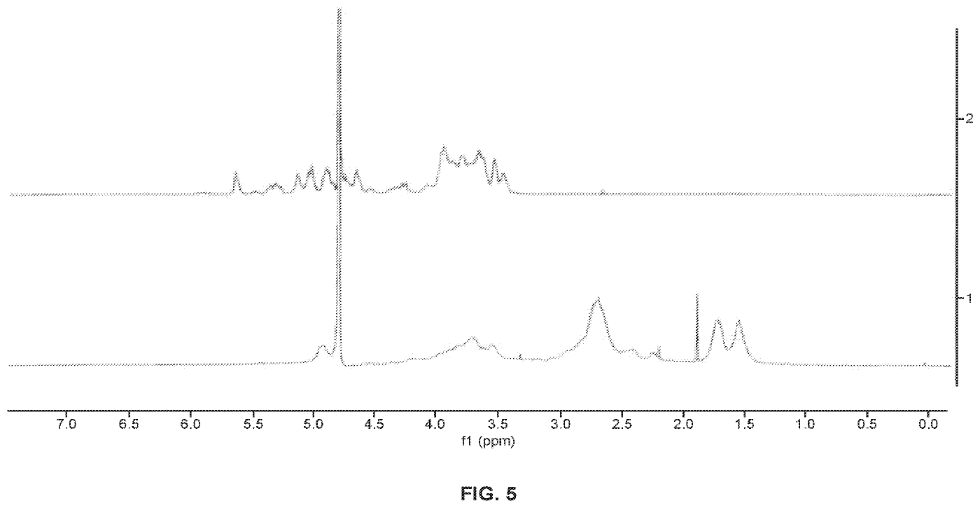

The synthesis of O-Dex is illustrated by the first chemical reaction in the series of reactions depicted in FIG. 4. Potassium periodate (1437 mg, 6.25 mmol) was added to a solution of Dextran 40 k (1000 mg, 6.25 mmol of glucose monomers) in milliQ water (20 mL). The reaction was vigorously stirred in the dark for 7 h at room temperature and then spin filtered twice using Amicon spin filters (MWCO=10 kDa) at 4000 g for 10 min at 4° C. with water washing. The resulting retentate was dialyzed for 1 day against water using a regenerated cellulose semi-permeable membrane (MWCO 3.5-5 kDa) followed by freeze-drying for 2 days to afford 0-Dex as a pale yellow solid (80% yield). ¹H NMR (400 MHz, D20) 6 (ppm)=6.02-4.85 (m, 0.4H), 4.71-4.25 (m, 0.5H), 4.05-3.41 (m, 1H), shown in the top of FIG. 5.

### Example 2: Synthesis of Spermine-Conjugated Dextran (Spe-Dex)

The synthesis of Spe-Dex from O-Dex is illustrated by the second and third chemical reactions in the series of reactions depicted in FIG. 4. O-Dex (790 mg, 4.94 mmol of glucose monomers) was dissolved in milliQ water (38 mL) and added to a solution of spermine (457 mg, 2.96 mmol) in borate buffer (19 mL, 0.1 M, pH=11) over 5 h via syringe pump. The resulting solution was gently stirred for 24 h at room temperature followed by the addition of NaBH₄ (359 mg, 9.48 mmol) under ice bath and stirring for 48 h at room temperature. An additional portion of NaBH₄ (359 mg, 9.48 mmol) was then added and stirring continued for 24 h under the same conditions. Crude product was dialyzed against water using Spectra-Par Float-A-Lyzer (MWCO=3.5-5 kDa) for 2.5 days followed by lyophilization for 2 days to yield Spe-Dex as a white fluffy solid. ¹H NMR (500 MHz, DzO) 6 (ppm)=3.66 (s, 1H), 2.74 (d, J=49.5 Hz, 1.5H), 1.86-1.39 (m, 1H), shown in the bottom of FIG. 5. The NMR suggests approximately every other glucose molecule of the dextran is conjugated to a spermine.

### Example 3: Spe-Dex Amine Quantification

The number of primary amines on Spe-Dex was quantified using the TNBS (2,4,6-Trinitrobenzenesulfonic acid) method with spermine as a standard. The reaction of TNBS with primary amines generates a chromophore whose intensity is directly proportional to the concentration of primary amines in the polymer. To create the spermine standard, 20 μL of freshly prepared aqueous TNBS solution (15 mg/mL) was separately added to tubes containing various amounts of spermine ranging from 0.04 to 0.2 μmol dissolved in 600 μL of water. 200 of bicarbonate buffer (0.8 M, pH=8.5) was added to each tube, the mixture vortexed for 1 min, and then incubated for 2 h at 37° C. Afterwards, 600 μL of 1 M HCl was added to each tube to quench the reaction, the mixture vortexed for 1 min, and then gently sonicated to remove any bubbles. 100 μL of sample was used per run and the absorbance was measured at 340 nm. The procedure was repeated using various masses of Spe-Dex and the number of amines was calculated using the equation of the standard curve and the absorbance of the labeled polymer. This method reported a count of 0.71 μmol of primary amine per milligram of Spe-Dex. For dextran molecule maintaining its starting molecular weight of 40 kDa, each resulting spermine-decorated dextran molecule would theoretically comprise approximately 33 spermines, 222 glucose units, and have a molecular weight of 46.71 kDa, in which approximately 1 out of every 6-7 glucose molecules is conjugated to a spermine, assuming no crosslinking between dextran molecules.

### Example 4: Polyplex Size Measurement

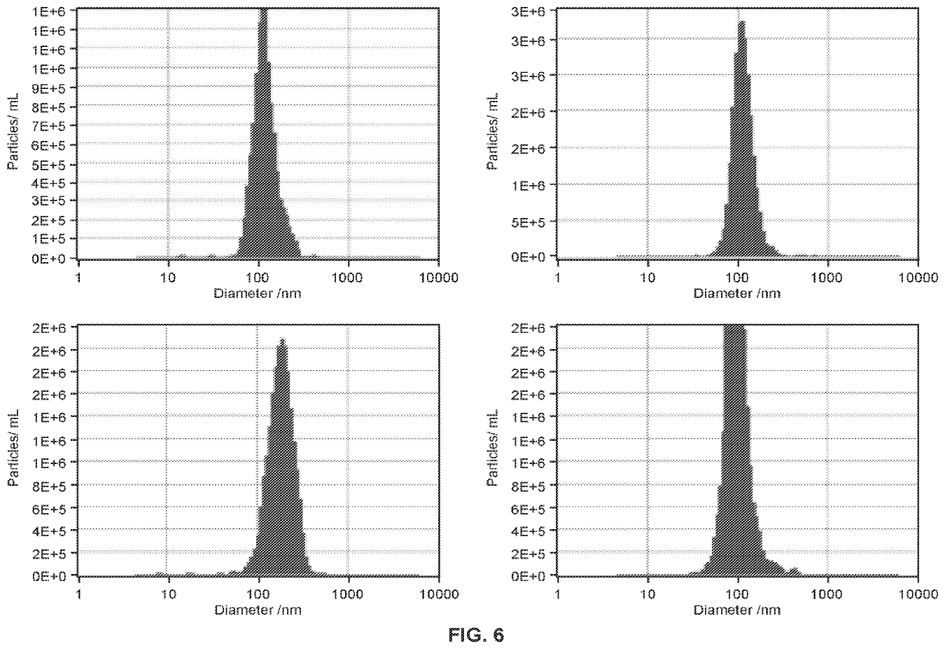

Spe-Dex/DNA polyplexes were prepared by vortexing 1 μg of GFP pDNA (50 μL, 20 μg/ml) with an equal volume of polymer solution at N:P ratios of 1:1, 1:2, 1:5, and 1:10 followed by 30 min of incubation at room temperature. 5 μL of each polyplex solution was then diluted to 1 ml with MQ water (or 10 mM NaCl) and the average size and zeta potential was measured using a ZetaView Particle Metrix NTA system. Particle size measurements of Spe-Dex/DNA polyplexes are depicted in FIG. 6 for polyplexes produced at N:P ratios of 1:1 (top-left), 1:2 (top-right), 1:5 (bottom-left), and 1:10 (bottom-right). Size measurements showed complexation of DNA at all ratios. Size and zeta for each ratio was as follows: 1:1=123.8 nm, 30.53 mV; 1:2=191.5 nm, 27.51 mV; 1:5=119.1 nm, −25.55 mV; 1:10=105.3 nm, −34.18 mV.

### Example 5: Gel Electrophoresis Assay

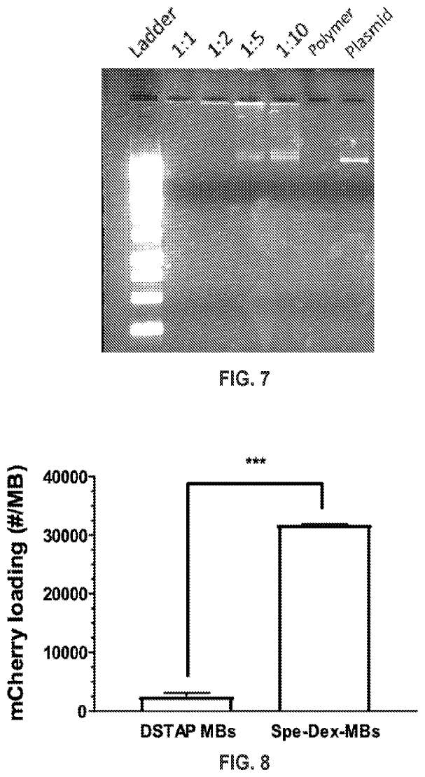

A 1% w/v agarose gel containing 0.8 μg/mL ethidium bromide (EtBr) was prepared in Tris-Acetate-EDTA buffer (TAE). Spe-Dex/DNA polyplexes were prepared at N:P ratios of 1:1, 1:2, 1:5, and 1:10 using the method of Example 4. 10 μL of each polyplex sample was diluted with 5 μL of 6×TriTrack DNA loading buffer and all 15 μL were loaded onto the gel. The gel was run at 70 mV for 30 min then at 100 mV for another 30 min and the DNA bands were visualized with an UV illuminator using a BioDoc-lt² Imaging System, the results of which are shown in FIG. 7. Gel assay verified complete complexation of DNA at N:P ratios of 1:1 and 1:2 as indicated by stationary bands but only partial complexation at ratios of 1:5 and 1:10, which showed bands of free DNA.

### Example 6: Spe-Dex Thiolation and FITC Labeling

Spe-Dex (5 mg, 3.55 mol NH₂) was dissolved with PBS 1×, 5 mM EDTA (0.5 ml). To this a 1 mg/ml aqueous solution of 2-iminothiolane HCL (4.88 mg, 35.45 μmol) was added dropwise with vigorous stirring. The resulting mixture was stirred for 1 h, dialyzed for 48 h (MWCO=3.5 kDa), and lyophilized for 48 h. Fluorescent labeling of Spe-Dex polymers was done using amine reactive 5/6-carboxyfluorescein succinimidyl ester (NHS-fluorescein).

Briefly, Spe-Dex polymer (5 mg) was dissolved in 1 ml of 1× borate buffer (50 mM, pH=8.5). NHS-fluorescein (5 mg, 10.562 μmol) was dissolved in DMF (0.5 ml) and added dropwise to the Spe-Dex solution with vigorous stirring. The reaction was stirred for 1 h, dialyzed for 48 h (MWCO=3.5 kDa), and lyophilized for 48 h.

### Example 7: Spe-Dex Thiol Quantification

The number of thiols on Spe-Dex was quantified using Ellman's Reagent (5,5′-dithiobis-(2-nitrobenzoic acid)) with cysteine as a standard. The reaction of Ellman's with thiols generates a chromophore whose intensity is directly proportional to the concentration of thiols in the polymer. To create the standard, 50 μL of cysteine at concentrations ranging from to 500 uM was added to 950 μL of 0.1 mM Ellman's dissolved in 0.1 M Tris-HCl pH 7.5 and vortexed briefly. The samples were then incubated for 2 min at RT. 100 μL of sample was used per run and the absorbance was measured at 412 nm. The procedure was repeated using various masses of Spe-Dex and the number of thiols was calculated using the equation of the standard curve and the absorbance of the labeled polymer. This method reported a count of 0.113 μmol of primary amine per milligram of Spe-Dex. This corresponds to the thiolation of approximately 1 out of every 6.28 primary amines and leaves approximately 27.89 primary amines free per dextran molecules, assuming no crosslinking between dextran molecules.

### Example 8: Microbubble Formulation

1,2-Distearoyl-sn-GLycero-3-Phosphocholine (DSPC), 1,2-Distearoyl-sn-GLycero-3-Phosphoethanolamine-N-[Methoxy(Polyethylene glycol)-2000] (DSPE-PEG2k), and 1,2-Distearoyl-sn-GLycero-3-Phosphoethanol amine-N-[Malemide(Polyethylene glycol)-2000] (Ammonium Salt) (DSPE-PEG2k-Mal) were each dissolved in 250 μL chloroform in molar ratios

of 90:5:5 and added to a 20 mL scintillation vial. Additional chloroform (2 mL) was added and the solution was dried with a rotary evaporator to create a lipid film around the vial. The film was further dried overnight in a vacuum desiccator to remove any residual chloroform. The resulting dry lipid films were re-suspended in PBS 1× buffer containing 10% propylene glycol and 10% glycerol and warmed to 70° C. The solution was sonicated at the same temperature until clear. The head space of the solution was filled with decafluorobutane gas and tip sonicated at 70% amplitude for 5 seconds. The microbubble solution was then drawn up with a syringe and centrifuged three times at 300 g for 3 min with PBS washing (pH=6.5, 1 mM EDTA). The average size and count of the microbubbles was obtained using a Beckman Coulter Multisizer 4. The measurements were done in triplicate with 2 μL of microbubble solution diluted into 10 ml of lsoton for each run. The average size of the maleimide-functionalized microbubbles was 2.981 μm+/−0.01 μm with a count of 2.22×10⁹+/−1.18×10⁸ MB s/mL.

### Example 9: Microbubble Conjugation with Spe-Dex

Spe-Dex polymer was conjugated onto microbubbles by covalently coupling the maleimide end-groups on the microbubble surface to the thiol groups on the Spe-Dex polymer. Briefly, microbubbles were first diluted down using PBS pH=6.5 w 1 mM EDTA to a concentration of 1.10×10 9 MBs/mL in a syringe. To this was added Spe-Dex polymer (10 mg/mL, PBS pH=6.5, 1 mM EDTA) at a maleimide:polymer molar ratio of 1:20. The suspension was rotated end-over-end at a speed of 20 rotations/min for 4 h at room temperature followed by washing 3× with PBS 1× at 300 g for 3 min. The average size of the Spe-Dex-deocrated microbubbles was 3.119 μm with a count of 3.42×10⁹ MBs/mL. Fluorescence microscopy was used to confirm conjugation of the F1-labeled Spe-Dex polymer onto the microbubble shell.

### Example 10: Quantification of Spe-Dex Conjugation to Microbubbles by Fluorescence Spectroscopy

To quantify the amount of F1-Spe-Dex conjugated to microbubbles, a standard curve was performed by diluting a 10 mg/mL solution of F1-Spe-Dex first in DMSO (1:20 dilution) and then in 5 mM NaOH (1:20 dilution). Serial dilutions were performed to obtain between 40 and 320 ng/mL F1-Spe-Dex in the wells (N=3). The linear regression of the calibration curve provided an equation of y=1.83×10⁷×-546.7 with an R² value of 0.9972. F1-Spe-Dex microbubble samples were prepared by diluting a volume of microbubble suspension in DMSO and 5 mM NaOH similarly to the standards. The F1-Spe-Dex MB sample was then sonicated for 20 s and vortexed to make sure that microbubbles were destroyed and that F1-Spe-Dex was homogenously distributed in solution. Solutions were excited at 488 nm and fluorescence intensity was read at 520 nm. Using the regression curve, the concentration of F1-Spe-Dex in the microbubble sample was measured to be 0.045 mg/mL. Per the calculations for spermine-dextran in Example 3, approximately 170,000 spermine-decorated dextran molecules are attached to each microbubble, comprising approximately 5.6 million spermines (4.7 million free spermines, assuming no crosslinking between dextrans) per microbubble.

### Example 11: Spe-Dex-MB DNA Loading

mCherry pDNA (0.403 μg/μL) was diluted to approximately 0.05 μg/μL with PBS 1× in a 1 mL syringe. A solution of Spe-Dex-MBs (20 μL, 2.4×10⁹ MBs/mL, 4.6×10⁷ total MBs) was slowly added to the syringe and the syringe was rotated at 18 rotations/min for 15 min then left upright at room temperature for 15 min. The microbubbles were centrifuged at 100 g for 1 min and the infranatant was discarded. The microbubbles were resized and had a concentration of 4×10⁸ MBs/mL with 1×10⁷ total MBs. The MB/DNA sample was then sonicated for 20 s and vortexed to make sure that microbubbles were destroyed and that DNA was homogenously distributed in solution. The absorbance of the sample was read at 260 nm using a Take 3 Multi-Volume plate and 2 μL of the sample in duplicate. The DNA concentration was calculated using a standard curve prepared from known concentrations of mCherry pDNA. Using this method, the number of pDNA per microbubble was determined to be 31,788 (N=4), which is a 10-fold, statistically significant increase in DNA-loading compared to cationic lipid (DSTAP) microbubbles (FIG. 8). Per the calculations of Example 10, approximately one molecule of pDNA was loaded for about every 148 spermines. Taking into account the average size of the microbubbles, a DNA loading capacity of approximately 0.028 μg/μm 2 was achieved, which is approximately 5.6× more than that achieved using PEI-conjugated microbubbles and at least 4.6× more than the loading achieved on microbubbles formed with cationic lipids, as reported in the literature. See, Sirsi et al. “Polyplex-Microbubble Hybrids for Ultrasound-Guided Plasmid DNA Delivery to Solid Tumors” J Control Release; 2012 Jan. 30; 157(2), which is hereby incorporated by reference in its entirety.

### Example 12: Formulation of CD44- or EpCAm-Targeted Microbubbles

Targeting moieties (such as hyaluronic acid for CD44 and anti-EpCAM antibodies) can also be conjugated onto the Spe-Dex-MBs. To target CD44, Spe-Dex-MBs were first diluted with PBS pH 6.5 with 1 mM EDTA to reach a concentration of 1.10×10⁹ MBs/mL. A 10 mg/mL solution of thiolated hyaluronic acid (MW=10,000 g/mol, CreativePegWorks) was added to the microbubble solution at a maleimide:polymer ratio of 1:5 (assuming all maleimides were free). The solution was rotated for 4 h and washed 3× with PBS 1× at 250 g for 2 min. To target EpCAM, Spe-Dex-MBs were conjugated with thiolated anti-EpCAM (Biolegend) antibody (Ab) using the same procedure but with using a malemide:antibody ratio of 2:1.

### Example 13: Spe-Dex-HA/Ab-MB Binding Experiments

A Circular Parallel Plate Flow Chamber Kit (GlycoTech Corporation) was used to confirm successful targeting of Spe-Dex-HA-MBs to CD44 expressed on HeLa cells. A 2% BSA in PBS solution (to prevent non-specific binding) was flowed through the chamber until it was saturated then vacuum was applied and HeLa cells growing in a 35 mm dish were placed on top of the chamber. 5×10⁶ MBs/mL in 2.5 mL were injected over 5 min at a flow rate of 450 μL/min using a syringe pump. After injection, the cells were washed with 1.3 mL of 2% BSA using the same flow rate and the vacuum was turned off and cells removed. Binding was confirmed by microscopy and the number of microbubbles in view was counted using Image) and compared to non-targeted Spe-Dex-MBs. Targeting showed 4134 microbubbles in view whereas non-targeting showed 815 microbubbles in view.

To test targeting to EpCAM, Spe-Dex-Ab-MBs were mixed with K562 cells in suspension. Spe-Dex-Ab-MBs with DNA were added to 1.65×10⁵ K562 cells (50 MBs/cell) and were mixed with gentle pipetting and incubated for 1 min. The MB/cell solution was then centrifuged at 300 g for 5 min to separate any non-targeted microbubbles and the supernatant was removed. The resulting pellet was resuspended in PBS and visualized under microscopy for any microbubbles targeted to cells. The procedure was repeated with non-targeted Spe-Dex-MBs as a control. The pellet with Spe-Dex-Ab-MBs showed 145 MBs in view whereas the pellet with Spe-Dex-MBs not conjugated with EpCAM antibody showed only 22 microbubbles in view.

### Example 14: Sonoporation of HeLa Cells Using Spe-Dex-HA-MBs

HeLa cells were sonoporated in a custom made apparatus consisting of a 35 mm dish whose lid had two holes drilled in it, one serving as an inlet for media and the second as an outlet for air. 24 h before transfection, HeLa cells were plated in this dish at 500,000 cells in 2 mL of complete EMEM, 10% FBS, 1% P/S. The day of transfection, 2×10⁷ Spe-Dex-HA-MBs were added to 9.64 μg of eGFP pDNA (N:P 1:5) and rotated for 15 min at a speed of 20 rotations per min then incubated for 15 min at room temperature. Microbubbles were then washed 1× by centrifugation at 250 g for 2 min and the infranatant was removed and kept for binding quantification. The media of the cells was then aspirated off and microbubbles were added dropwise at a final concentration of 20 MBs/cell. The well was then flipped upside down and incubated for 10 min to allow for the microbubbles to target the cells. Afterwards the well was flipped back and the lid was parafilmed on to prevent leaking. The well was filled with OptiMEM and then flipped back upside down. US coupling gel was applied to the bottom of the well and the cells were sonoporated at 1 W/cm², 50% DC for 60 seconds while moving the transducer back and forth across the well. 4 h post sonoporation, the OptiMEM was aspirated off and replaced with 2 mL of complete EMEM. Transfection was confirmed 3 days post sonoporation using fluorescence microscopy and assessed via flow cytometry. Sonoporated cells show a 14% increase in FITC signal as compared to control cells.

### Example 15: Sonoporation of JeKo-1 Cells Using Spe-Dex-ROR1-MBs

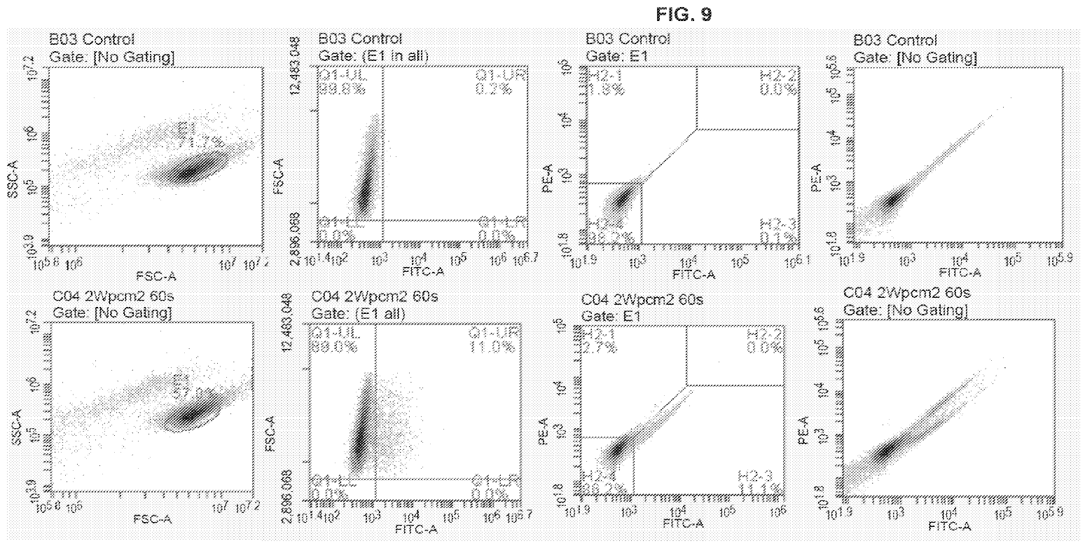

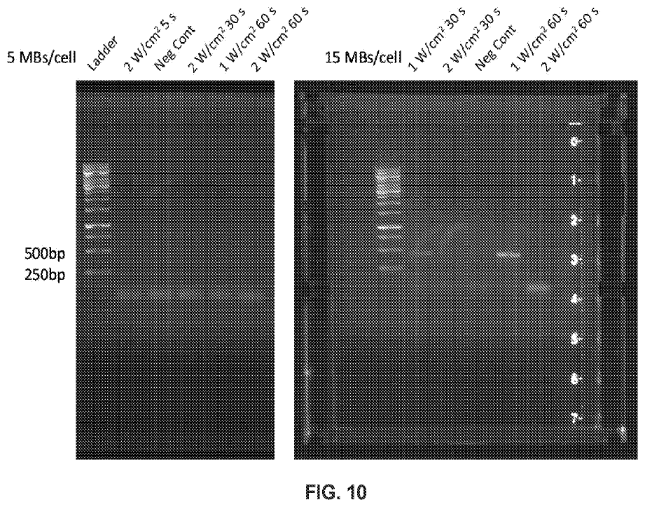

3×10⁷ Spe-Dex-ROR1-MBs were added to 28.93 μg of eGFP pDNA (N:P 1:10) and rotated for 15 min at a speed of 20 rotations per min then incubated for 15 min at room temperature. 2.5×10⁶ JeKo-1 cells were spun down, resuspended in 1 mL of RPMI, and 500,000 cells were added to the control well of a 12 well plate. Microbubbles were added to the remaining cells and the suspension was rotated for 15 min to allow microbubble targeting to ROR1 on the cells (approximately 15 MBs/cell). Afterwards, the suspension was diluted to mL and 2.5 mL was added to each well. The plate was placed on top of a heat bath set to 35° C. and wells were sonoporated at either 1 or 2 W/cm² with 50% DC for either 30 or 60 seconds. 2.5 h post sonoporation, cells were spun down and resuspended in 1 mL complete RPMI. After 3 days, cells were collected for flow cytometry (FIG. 9). Gating for live cells showed a maximum increase of 11% GFP signal for sonoporated cells (top) compared to negative control samples (bottom; 2 W/cm², 50% DC, 60 s). Plotting FITC vs PE channels on the cytometer allowed for separation between autofluorescence and true GFP signal, which further confirmed transfection. However, gating for live cells only removed the autofluorescence signal. To further confirm transfection, DNA was isolated from both control and sonoporated cells using the TRIzol method. The isolated DNA was amplified using PCR and the forward and reverse primers of eGFP. The amplified DNA along with a DNA ladder was run on a 1% agarose gel stained with EtBr and visualized with a BioDoc-lt² Imaging System (FIG. 10). The gel shows GFP bands for some sonoporated samples but not all, even though flow cytometry showed all sonoporated samples as GFP positive. This may be because the GFP protein is still in the cell while the plasmid has been degraded.

### Example 16: Selectively Transfecting T-Cells In Vivo with Spe-Dex MBs Loaded with mCherry Encoding Plasmid

In Vivo Transfection in Rats:

In vivo expression is optimized to maximize the fraction of cells expressing the gene and gene expression. In vivo transfection may rely on transfection conditions optimized in vitro, accounting for ultrasound attenuation by intervening tissues and blood flow that decreases exposure time of microbubble/cell complexes. T-cells are transfected by insonating the spleen where 30% of T-cells reside and a large blood space, such as the cava or heart. First, the spleens of 6 rats, where T-cells are stationary, are sonoporated. The optimal microbubble formulation and count assuming 10⁶ T-cells/mL and 80 mL blood/kg body weight to achieve the optimal microbuble/cell ratio is injected intravenously. The experiment is begun with the optimal ex vivo ultrasound exposure 10 minutes after microbubble administration and in vivo transfection by measuring the fraction of circulating T-cells expressing the gene at 72 hours is assessed and gene expression is quantified as described above. Spleens are analyzed post mortem. The spleens are first assessed for burns. Half are then homogenized and half are fixed. The homogenate is assessed by fluorescence microscopy and flow cytometry after adding FITC-aCD3 to stain T-cells. H&E and immunohistochemistry staining with FITC-aCD3 are done to assess for injury and look for co-localization of mCherry and FITC. The ultrasound SATA energy is then increased for the next 6 rats and in vivo transfection is re-assessed as before. The exposure that maximizes the fraction of transfected T-cells and gene expression with no tissue injury, is then be used to optimize exposure time when insonating blood.

The same microbubble formulation and count used for spleen transfection is be infused. The optimal exposure time determined ex vivo and used for spleen transfection, is used as a starting point and then exposure time is shortened as ultrasound power is increased to maintain a constant SATA as the inferior vena cava and portal vein at the level of the kidneys before they enter the liver is insonated. In vivo transfection is assessed by measuring the fraction of circulating T-cells expressing the gene at 72 hours and quantifying gene expression as described above. Spleens are also evaluated by assessing spleen homogenate and tissue fluorescence as described above. These experiments are done in 6 rats for each iteration and the differences analyzed statistically as described above.

The data obtained from these optimized in vivo T-cell transfection experiments may be used to determine whether spleen or blood sonoporation is a superior transfection method. Both methods may be used for the in vivo transfection experiments described elsewhere herein, unless explicitly stated otherwise. A method which produces <15% transfection than the other may be deemed less effective.

Gene Transfection Half-Life In Vivo:

For calculating gene transfection half-life in vivo, rats are transfected using the optimal ultrasound exposure and transfection method determined above. 6 rats are sacrificed at 1, 2, 4, 7, and 14 days after sonoporation and the fraction of transfected T-cells is determined as in blood and spleen described above. The average fractional T-cells transfected and gene expression levels are plotted over time and gene expression half-life is determined. The change over time is evaluated statistically using a 1-way ANOVA and data points compared using unpaired Student's t-test. Gene expression half-life may be calculated following ex vivo transfection of 4 mL of rat blood using the optimal parameters determined for ex vivo transfection if the fraction of transfected T-cells are low after in vivo transfection. 2×10⁶ T-cells are enriched and injected immediately following sonoporation in litter mates.

Maximizing fraction of in vivo transfected T-cells: 30 rats undergo several transfection sessions (microbubble infusion and sonoporation), where each 6 rats will be treated every T½/3, T½/2, T½, 1.5×T½ or 2×T½. Six additional rats serve as control. The rats are infused with mCherry-loaded and non-specific IgG-labeled microbubbles and sonoporated at the shortest interval of the experimental group. One day following the last treatment 200 μL of blood is analyzed for fraction of transfected T-cells, and then again at 2, 4, 7, and 14 days and at 3 times the T½, when animals will be sacrificed and the blood and spleen analyzed for T-cell transfection as described above. The average number of transfected T-cells is plotted for each treatment group to determine the blood count of transfected T-cells and analyzed for statistical significance using a 2-way analysis of variance where time and treatment groups serve as the independent variable. The cell count is expected to be greatest with shorter treatment intervals, to plateau when the interval is equal to T½, and to decrease with intervals >T½.

Validation of Activation and CAR Expression in Transfected Rat T-Cell In Vitro:

T-cells are transfected in blood using the optimal conditions for ex vivo transfection and transfected T-cells are isolated and counted. Transfected T-cells are then allowed to interact with Fc-tagged CD19 protein that will be coated at the bottom of a 96 well culture plate using 1×10⁵ CAR T-cells added to each well containing the optimal culture media. IL2 and IFN-gamma cytokine release is measured by enzyme-linked immunosorbent assay (ELISA) in the supernatant after 30 min, 2, 12, 24 and 48 h. At these time points, CAR T-cells are also collected and flow cytometry is performed to quantify markers for T-cell activation, memory and exhaustion by assaying CD69, CD62L, PD1, LAG3, and TIM3 expression. T-cells are retrieved from non-transfected blood and T-cells transfected with mCherry serve as negative controls. Wells coated with other proteins are used to demonstrate the CAR-target specificity. Experiments are done in triplicate.

Validation of Biological Cytotoxicity of CAR Expressing Rat T-Cells In Vitro:

Using EasySep™ Rat T-Cell and B-cell separation kits (STEMCELL Technologies) of normal rat blood, 1×10⁵ CAR T-cells are cultured and transfected ex vivo with an equal number of B-cells to validate biological cytotoxicity of CAR-T cells. B-cells cultured with non-transfected T-cells serve as negative controls. Flow cytometry with AF488-CD19 antibody and PI staining is used to assess the number of live B-cells before and at 30 min, 2, 12, 24 and 48 h after mixing with CAR T-cells. B-cell lysis is also quantified using CytoTox 96 ® (Promega™), a non-radioactive colorimetric cytotoxicity assay. Briefly, supernatant is collected and transferred to a new 96-well plate and the CytoTox reagent added. Stop solution is added at to end the reaction. Absorbance will be measured at 490 nm using a microplate reader. Spontaneous lactate dehydrogenase (LDH) release of B-cells is measured from the CAR T-cell free wells and maximal release is measured from Triton X-100 treated samples. Percent cytotoxicity is calculated as 100×[(Experimental LDH release−Spontaneous LDH release)/(Maximal LDH release−Spontaneous release)]. Experiments are done in triplicate.

Validation of In Vivo T-Cell Transfection and CD19+ Cell Depletion:

T-cells are transfected in vivo in 12 rats with either CD3-targeted (n=6) or non-targeted microbubbles (n=6) loaded with the CD19 CART plasmid. Experimental conditions that yield the largest fraction of transfected T-cell, likely following multiple treatments, are followed. 200 μL of blood is collected at 12, 24, 48, and 72 h and analyzed for B-cell depletion. AF488-aCD19 is added to the blood sample and B-cells assessed for viability using flow cytometry and PI staining. Liver, spleen, bone marrow, lymph nodes, and lung are collected. Samples are homogenized and the remainder stained with H&E and immunohistochemistry (IHC) using fluorescently labeled anti-CD19 antibody. Homogenates are assessed for quantifying the number of transfected T-cells as described above, and live B-cells are assessed by flow cytometry using AF488-aCD19, AF-647-aCD3 and SYTOX™ Orange.

Validation of Activation, B-Cell Cytotoxicity of CAR Expressing ALL T-Cells and No Off-Target CAR Transfection In Vitro:

To assess CAR T-cell activation and biological activity, but with human blood from acute lymphoblastic leukemia (ALL) patients, the T-cells of healthy and ALL patients are transfected ex vivo and therapeutic efficacy is validated using a mouse model of a human B-cell ALL xenograft. Using blood samples from ALL patients, T-cells are transfected ex vivo using the optimized conditions and transfected T-cells are counted. Transfected T-cells are allowed to interact with Fc-tagged CD19 protein as with the rat blood and IL2 and IFN-gamma cytokine release is measured in the supernatant by ELISA after 30 min, 2, 12, 24 and 48 h. At these time points, CAR T-cells are collected and flow cytometry is performed to quantify markers for T-cell activation, memory and exhaustion as with the rat blood. T-cells retrieved from non-transfected blood and T-cells transfected with mCherry serve as negative controls. Wells coated with other proteins are used to assess CAR-target specificity. Experiments are done in triplicate.

Validation of Biological Cytotoxicity, of CAR Expressing Human T-Cells In Vitro:

Using the buoyancy technique, T-cells and B-cells are isolated separately using CD3 or CD19 targeted microbubbles. T-cells are transfected ex vivo and then cultured and expanded for 72 hours. T-cells are then isolated and 1×10⁵ live CAR T-cells are mixed with B-cells at a 1:1, 5:1, 10:1 B- to T-cell ratio to assess for biological cytotoxicity. Non-transfected T-cells and transfected T-cells mixed with WBC (CD19−) serve as negative controls. Released IL2 and IFN-gamma in the supernatant is measured by ELISA, and flow cytometry with AF488-CD19 antibody and PI staining is used to count the number of live B-cells before and at 30 min, 2, 12, 24 and 48 h after mixing with CAR T-cells. B-cell lysis is quantified using CytoTox 96 ® (Promega™), as described above. Percent cytotoxicity is measured and calculated. Experiments are done in triplicate.

Human Xenograft Model of B-ALL:

Xenografts of 5×10⁶ pre-B-ALL NALM-6 cells are performed in SCID mice. 78, 6-8 weeks old mice are radiated with a sub-lethal dose of 250 cGy over the entire body to improve engraftment, which is assessed in 3 mice each at 6 weeks and 8 weeks by counting the number of infiltrated human leukemia cells in peripheral blood post RBC lysis, as well as in the liver, spleen, and bone marrow nucleated cells using flow cytometry after staining with anti-human PE-CD19 and anti-human PE-CD45. Additional samples are also stained with non-specific antibodies labeled with PE to set gates for positive staining. T-cells isolated from ALL blood are transfected ex vivo to express the CD19-CAR as described above and cultured for 72 hours. 2×10⁶ live CAR T-cells are isolated and injected intravenously in 18 SCID mice at 6 (N=9) or 8 weeks (N=9) after ALL B-cell inoculation. 2×10⁶ non-transfected T-cells isolated from ALL patient blood are injected intravenously in 18 SCID mice at 6 (N=9) or 8 (N=9) weeks after ALL B-cell inoculation as control. 3 mice from each group are bled at 24, 48, and 72 h after T-cell administration. AF488-aCD19 is added to the blood sample and B-cells are assessed for viability using flow cytometry and PI staining. Liver, spleen, bone marrow, lymph nodes, and lung are collected and IHC performed to detect the presence of CD19+ cells. The survival of treated mice (N=18) vs control (N=18) is also monitored.

### Example 17: Sonoporation of B-Cells with Spe-Dex-MBs

Spermine-dextran microbubbles (Spe-Dex-MBs) are loaded with the gene encoding for CD154 and targeted to ROR-1 by attaching anti-ROR-1 antibodies to the micobubble surface, as described elsewhere herein. Using luciferase as a surrogate gene, microbubble loading, dose and ultrasound parameters are optimized to maximize gene expression while minimizing cell death in vitro. T-cell activation, proliferation and malignant B-cell killing is assessed before and after transfection in vitro and then in vivo using a murine model of B-cell malignancy employing the formulation and ultrasound conditions optimized in the in vitro experiment. Minutes after microbubbles are given systemically, they should attach to circulating malignant B-cells, which are then transfected by exposing the MB/cell complexes to an ultrasound field that is delivered using a clinical ultrasound system at or below FDA-approved transmit power. Production of CD154 should begin shortly after. When enough cells are transfected, they will induce T-cell activation to begin malignant B-cell killing.

### Example 18: Synthesis of TFA2-Spermine

The synthesis of TFA2-Spermine is depicted in FIG. 11. Ethyltrifluoroacetate (1.47 mL, 12.36 mmol) and water (0.089 mL, 4.94 mmol) were added to a solution of spermine (500 mg, 2.47 mmol) in acetonitrile (8 mL), and the resulting mixture was refluxed at 90° C. for 3 h. After this time, the reaction mixture was cooled down to room temperature to form a white precipitate and cold DCM (2 mL) was gradually added to complete precipitation. The solid was filtered, washed with cold DCM, and dried under vacuum to yield TFA2-Spermine as a white solid. ¹H NMR (400 MHz, D₂O) δ (ppm)=3.41 (t, J=6.8 Hz, 4H), 3.10-3.01 (m, 8H), 2.00-1.90 (m, 4H), 1.78-1.70 (m, 4H).

### Example 19: Synthesis of BOC2-Spermine

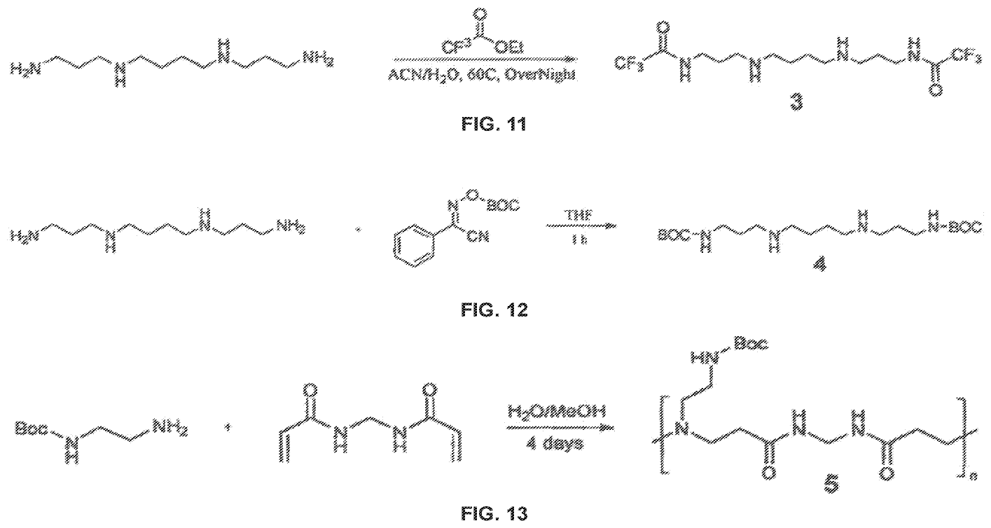

The synthesis of BOC₂-Spermine is depicted in FIG. 12. Spermine (70 mg, 0.346 mmol) was dissolved in THF (1.05 mL) under an argon atmosphere and cooled using an ice bath. To this solution, BOC-ON (170 mg, 0.692 mmol) in THF (2.11 mL) was added dropwise and the reaction mixture was stirred in the ice bath for 2 min then at room temperature for 1 h. The reaction was then quenched with saturated sodium carbonate (3.15 mL) and extracted 3× with DCM (6 mL each). The organic layer was dried with sodium sulfate, evaporated, and purified with PuriFlash using a 12 g 15 μm silica column and 5% of 28% NH₄OH in ethanol as eluent to afford BOC2-Spermine as a viscous liquid. ¹H NMR (500 MHz, CDCl₃) δ (ppm)=(br, 1H), 3.2-3.1 (m, 2H), 2.65 (t, J=6.4 Hz, 2H), 2.6-2.5 (m, 2H), 2.4-2.2 (br, 1H), 1.64 (t, J=6.4 Hz, 2H), 1.5-1.4 (m, 2H), 1.40 (s, 9H).

### Example 20: Synthesis of Poly Diaminoethane (PolyDAE)

The synthesis of PolyDAE is depicted in FIG. 13. BOC-DAE with methylene bisacrylamide was carried out as a proof of concept. BOC-DAE (100 mg, 0.624 mmol) and methylene bisacrylamide (96 mg, 0.624 mmol) were added to a round-bottom flask, flushed with argon, and dissolved in degassed 9:1 v/v MeOH/H₂O solvent mixture (0.62 mL). The reaction mixture was stirred in the dark at 60° C. under argon balloon for 4 days. Afterwards, the reaction was precipitated in 25 mL of cold diethyl ether. The solid was filtered off, dried, and then dissolved in 97.5:2.5 v/v TFA/H₂O (1 mL) and stirred for 45 min to remove BOC protecting groups. The product was then precipitated again in 25 mL cold diethyl ether, filtered, and dried to afford PolyDAE as a viscous liquid. ¹H NMR (500 MHz, D₂O) δ (ppm)=4.54 (d, J=4.0 Hz, 2H), 3.58-3.29 (m, 10H), 2.72 (dt, J=48.1, 6.5 Hz, 4H).

### Example 21: Synthesis of Polyspermine

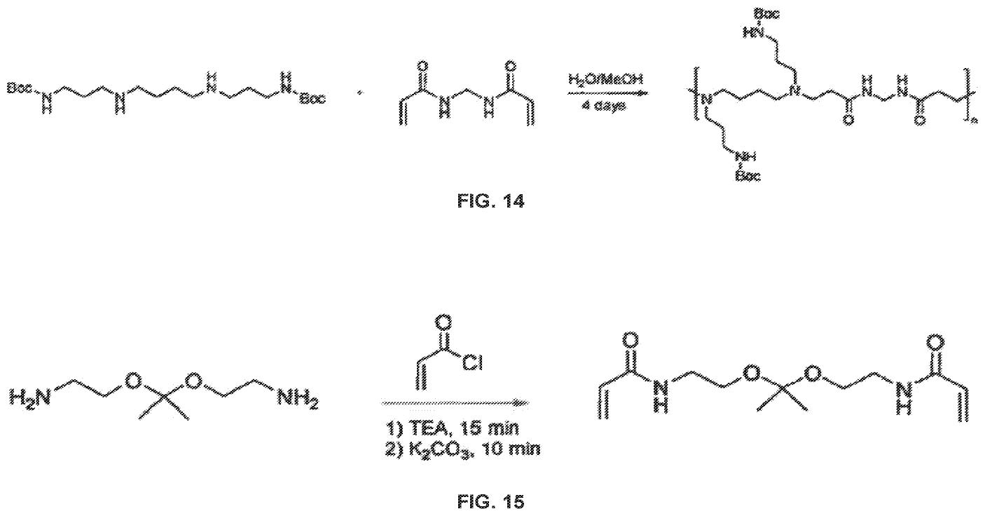

The synthesis of polyspermine is depicted in FIG. 14. BOC2-Spermine (200 mg, mmol) and methylene bisacrylamide (77 mg, 0.497 mmol) were added to a round-bottom flask, flushed with argon, and dissolved in degassed 9:1 v/v MeOH/H₂O solvent mixture (0.75 mL). The reaction mixture was stirred in the dark at 60° C. under argon for 4 days. Afterwards, the reaction was precipitated into 30 mL cold ether. The solid was filtered off, dried, and then dissolved in 1 mL of 95:5 v/v TFA/H₂O and stirred for 30 min to remove BOC protecting groups. The crude product was precipitated into 30 mL of cold ether and dried under vacuum. The product was further purified by dialyzing it for 1 day (MWCO=1 kDa) followed by lyophilization to afford polyspermine as a white powder (33 mg). ¹H NMR (400 MHz, D₂O) δ (ppm)=4.54 (s, 2H), 3.00 (p, J=9.6, 8.7 Hz, 8H), 2.83-2.60 (m, 8H), 2.49 (t, J=7.4 Hz, 4H), 1.90 (q, J=8.2, 7.7 Hz, 4H), 1.52 (s, 4H).

### Example 22: Synthesis of Bisacrylamide Ketal

The synthesis of Bisacrylamide ketal is depicted in FIG. 15. Bisphthalimide ketaldiamine (1000 mg, 2.37 mmol) was added to a round-bottom flask fitted with a reflux condenser. 6 M NaOH (6.41 mL) was added and the reaction mixture was refluxed overnight at 100° C. to remove the phthalimide protecting groups. Unprotected ketaldiamine was then extracted with a 1:1 v/v mixture of CHCl₃/iPrOH (3×, 13 mL). Ketaldiamine (384 mg, 2.37 mmol) was then added to a RB flask in an ice bath and dissolved with 6 M NaOH (11.5 mL). Triethylamine (14.9 mL, 107 mmol) and acryloyl chloride (2.87 mL, 35.5 mmol) were added in small, alternating portions to maintain the pH above 8. The resulting mixture was stirred for min followed by the addition of cold 10% K₂CO₃ solution (24 mL) and stirring for 10 more min. The crude product was extracted with ethyl acetate (3×, 12 mL) and purified on a silica column using first 1:1 ethyl acetate/hexane as eluent then 100% ethyl acetate. The final product was then crystallized with ethyl acetate (105 mg, 21.4%).

### Example 23: Synthesis of Phthalimide-Spermine

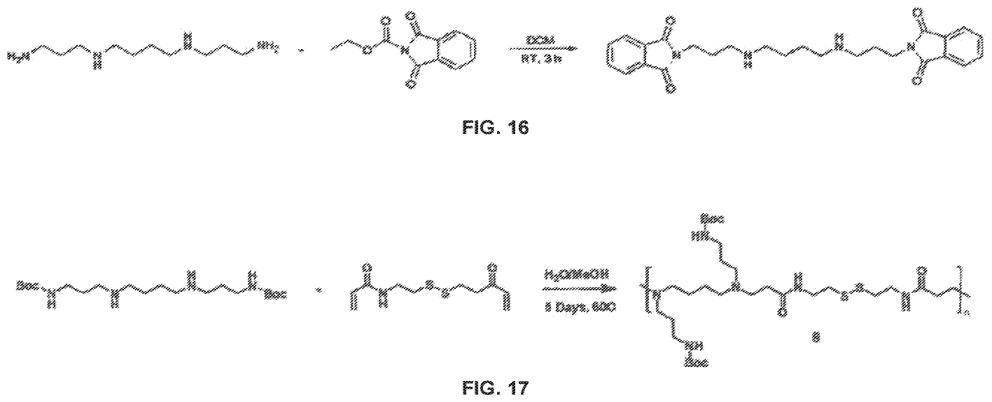

The synthesis of Phthalimide-Spermine is depicted in FIG. 16. Spermine (400 mg, 1.98 mmol) and N-carbethoxyphthalimide (867 mg, 3.95 mmol) were added to a round-bottom flask and dissolved in DCM (7 mL). The reaction mixture was stirred for 3 h then evaporated to dryness. The residue was purified over a silica column using first 10:1 DCM/MeOH as eluent then 2:1 DCM/MeOH to give Phthalimide-Spermine as a pale yellow solid. Thin layer chromatography (TLC) R_{f}=0.2 (2:1 v/v DCM/MeOH). ¹H NMR (400 MHz, CDCl₃) δ (ppm)=7.85-7.63 (m, 8H), 3.72 (t, J=6.8 Hz, 4H), 2.68-2.52 (m, 8H), 1.85 (p, J=7.0 Hz, 5H).

### Example 24: Synthesis of Polyspermine Cystamine Bisacrylamide (Polyspermine CBA)

The synthesis of polyspermine CBA is depicted in FIG. 17. CBABOC₂-Spermine (341.9 mg, 0.849 mmol) and cystamine bisacrylamide (221.13 mg, 0.849 mmol) were added to a RB flask, flushed with argon, and dissolved in degassed 9:1 v/v MeOH/H₂O solvent mixture (1.28 mL). The reaction mixture was stirred in the dark at 60° C. under argon balloon for 5 days. After this time, 1 mL of 95:5 v/v TFA/H₂O was added and the resulting mixture was stirred for 45 min to remove BOC protecting groups. The crude product was dialyzed for 1 day (MWCO=1 kDa) followed by lyophilization to afford Polyspermine CBA as a white powder (66.7 mg). ¹H NMR (400 MHz, D₂O) δ (ppm)=3.52 (t, J=6.5 Hz, 4H), 3.26 (t, J=7.2 Hz, 3H), 3.12-2.96 (m, 11H), 2.82 (t, J=6.5 Hz, 4H), 2.65 (t, J=7.2 Hz, 4H), 2.09-1.97 (m, 4H), 1.76-1.63 (m, 4H).

1. A microbubble composition for delivering a payload to one or more cells, the microbubble comprising a plurality of spermine-decorated microbubbles, wherein the microbubbles each comprise a gas core encapsulated by a surfactant shell, and wherein a plurality of spermine molecules are associated with the external surface of the surfactant shell of each microbubble.

2-14. (canceled)

15. The microbubble composition of claim 1, wherein the gas core comprises a perfluorocarbon.

16. The microbubble composition of claim 15, wherein the perfluorocarbon is decafluorobutane.

17. The microbubble composition of claim 1, wherein the surfactant shell comprises lipids.

18. The microbubble composition of claim 17, wherein the lipids are phospholipids.

19. The microbubble composition of claim 18, wherein the phospholipids comprise one or both of 1,2-Distearoyl-sn-Glycero-3-Phosphocholine (DSPC) and 1,2-Distearoyl-sn-Glycero-3-Phosphoethanolamine (D SPE) lipids.

20. The microbubble composition of claim 1, wherein the surfactant shell comprises PEGylated molecules.

21. The microbubble composition of claim 1, wherein the surfactant shell comprises a plurality of reactive groups exposed on the external surface of the surfactant shell.

22. The microbubble composition of claim 21, wherein the reactive groups are maleimides.

23-24. (canceled)

25. The microbubble composition of claim 1, wherein the plurality of spermine molecules on each microbubble are associated with a plurality of payload molecules.

26. The microbubble composition of claim 25, wherein the payload molecules comprise proteins.

27. The microbubble composition of claim 25, wherein the payload molecules comprise nucleic acids.

28. The microbubble composition of claim 27, wherein the nucleic acids comprise DNA, optionally wherein the DNA is a plasmid.

29. (canceled)

30. The microbubble composition of claim 1, wherein the microbubbles further comprise targeting molecules on the external surface of the surfactant shell, the targeting molecules being configured to bind the one or more cells.

31. The microbubble composition of claim 30, wherein the targeting molecules comprise antibodies.

32. The microbubble composition of claim 1, wherein the average microbubble size of the microbubble composition is between about 1 μm and 10 μm.

33. The microbubble composition of claim 32, wherein the average microbubble size is between about 1 μm and about 5 μm

34. The microbubble composition of claim 33, wherein the average microbubble size is about 3 μm.

35-39. (canceled)

40. A method of making a microbubble composition for delivering a payload to one or more cells, the method comprising mixing a solution comprising spermine molecules with a solution of microbubbles, wherein the microbubbles each comprise a gas core encapsulated by a surfactant shell.

41. (canceled)

42. The method of claim 40, wherein the solution comprising spermine molecules comprises spermines conjugated to polymers.

43. The method of claim 42, wherein the polymers are scaffold polymers, multiple spermine molecules being covalently coupled to the scaffold polymer.

44-45. (canceled)

46. The method of claim 43, wherein the scaffold polymer is dextran.

47-64. (canceled)

65. The method of claim 40, further comprising mixing in a solution comprising payload molecules.

66-69. (canceled)

70. The method of claim 65, wherein the payload molecules comprise proteins.

71. (canceled)

72. The method of claim 70, wherein the nucleic acids comprise DNA, optionally wherein the DNA is a plasmid.

73-76 (canceled)

77. The method of claim 40, further comprising
mixing a solution comprising targeting molecules with a solution comprising the microbubbles.

78-81. (canceled)

82. The method of claim 77, wherein the targeting molecules are antibodies.

83. (canceled)

84. A method of delivering a payload to one or more cells using sonoporation, the method comprising exposing the one or more cells to a plurality of spermine-decorated microbubbles and then exposing the one or more cells to an ultrasound stimulus configured to sonoporate the one or more cells.

85. The method of claim 84, wherein the ultrasound is delivered at about 1-5 W/cm², optionally with 50% between 20%-90% duty cycle.

86-87. (canceled)

88. The method of claim 84, further comprising using ultrasound to visualize the microbubbles prior to delivering the ultrasound stimulus configured to sonoporate the one or more cells, wherein the intensity of the ultrasound used to visualize the microbubbles is less than the intensity of the ultrasound stimulus.

89-95 (canceled)

96. A microbubble composition for delivering a payload to one or more cells, the microbubble comprising:
a) a plurality of spermine-decorated microbubbles, wherein the microbubbles each comprise a gas core encapsulated by a surfactant shell, and wherein a plurality of spermine molecules are associated with the external surface of the surfactant shell of each microbubble;
b) a payload bound to the plurality of spermine molecules; and
c) a targeting molecule conjugated to the exterior of said microbubble.
